(19) United States
(12) Patent Application Publication
(10) Pub. No.: US 2022/0226281 A1
(43) Pub. Date: Jul. 21, 2022

(54) COMPOUNDS FOR USE IN ANTI-CANCER IMMUNOTHERAPY

(71) Applicants: Sirenas LLC, San Diego, CA (US); The Scripps Research Institute, La Jolla, CA (US)

(72) Inventors: Steven Bruce Cohen, San Diego, CA (US); Dan Lu, San Diego, CA (US); Farhana Barmare, San Diego, CA (US); Ippei Usui, San Diego, CA (US); Venkata Rami Reddy Macherla, San Diego, CA (US); Elizabeth Paige Stout, San Diego, CA (US); Jacob Neal Beverage, San Diego, CA (US); Eduardo Esquenazi, San Diego, CA (US); Peter A. Jordan, San Diego, CA (US); Phil S. Baran, San Diego, CA (US); John R. Teijaro, San Diego, CA (US)

(21) Appl. No.: 17/614,118

(22) PCT Filed: May 28, 2020
(86) PCT No.: PCT/US2020/034991

§ 371 Date: Nov. 24, 2021

Related U.S. Application Data
(60) Provisional application No. 62/854,903, filed on May 30, 2019.

(51) Int. Cl. A61K 31/365; A61K 31/395; A61P 35/00; A61K 45/06

(52) U.S. Cl. CPC A61K 31/365; A61K 45/06; A61P 35/00; A61K 31/5545

(57) ABSTRACT

The present disclosure relates to the reversal of T cell exhaustion using aplysiatoxin analogs or PKC theta agonist compounds for anti-cancer immunotherapy. The treatment with aplysiatoxin analogs or PKC theta agonist compounds improve the anti-tumour activity of a T cell, inducing a NFAT-dependent T cell activation, increasing the pool of immune-checkpoint inhibitor responsive T cells, increasing lymphocyte infiltration and increasing the population of activated CD4+ and/or CD8+ cells. The methods of the present disclosure provide treatment of tumors and infections.

### BACKGROUND OF THE INVENTION

### Field

The present disclosure relates to the reversal of T cell exhaustion using aplysiatoxin analogs and PKC theta agonist compounds for anti-cancer immunotherapy.

### Description of the Related Art

T cells are a type of white blood cells that play a key role in cell-mediated immunity and fighting cancer. T cell exhaustion is a state of T cell dysfunction that arises during many chronic infections and cancer and can prevent optimal control of infection and tumors. T cells that lose ability to respond/eradicate tumors take on an exhaustion phenotype, which includes high levels of inhibitory receptors, decreased effector cytokine production and cytolytic ability. This loss of functional and phenotypic features occur in a stepwise method. The function of production of interleukin-2 (IL-2) is affected first, followed by tumor necrosis factor-α and interferon-γ. Lastly, the T-cells might undergo apoptosis due to apoptotic factor expression and failure to respond to IL-7 and IL-15 (regulators of T-cell homeostasis). Serine/threonine-specific protein kinase C-theta (PKC theta) is a kinase instrumental in activating a wide range of signaling cascades in T cells. It plays an important role in T cell activation, proliferation and differentiation.

Although the mechanistic details of T cell exhaustion are still being clarified, it has become clear that both extrinsic negative regulatory pathways and cell-intrinsic regulatory pathways (such as PD-1) have key roles in exhaustion. Blocking the PD-1 pathway partially reinvigorates exhausted T cells in preclinical models and the strategy of checkpoint inhibition via PD/PD-L1 blockade has demonstrated impressive results in certain tumors. Response to PD-1 checkpoint therapy depends on balance of overall tumor burden and reservoir of responsive/re-invigorated CD8+ T cells. Therapies that reverse T cell exhaustion over and above PD/PD-L1 therapy have the potential to tip the balance towards invigoration of CD8+ T cells to match tumor burden towards eradication and durable response.

In many cancers, defective T cell function is now considered a main event allowing tumor growth and disease. Restoring tumor infiltrating T cell (TIL) function through blockade of inhibitory “checkpoint” receptors such as CTLA-4 or PD-1 can achieve complete and persistent responses in approximately 20% of patients in some cancers. This has led to the recent approval of checkpoint therapies in several cancer types and sparked enormous interest in finding ways to render the remaining ˜80% of patients responsive to cancer immunotherapies. Accordingly, a need exists for therapies that reverse T cell exhaustion due to their potential role in improving prognosis and survival in cancer patients.

### SUMMARY OF THE INVENTION

Disclosed herein is a method of reversing T-cell exhaustion in a subject, the method comprising administering to the subject a compound of Formula (I):

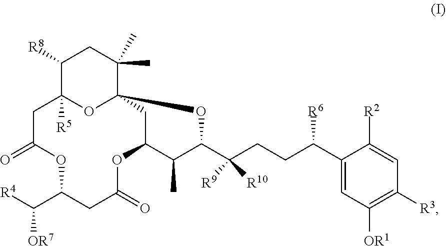

or a pharmaceutically acceptable salt or prodrug thereof, wherein:

R¹ may be —H, —C(═O)CH₃, —CH₂C₆H₅, —CH₂—O—CH₂C₆H₅, C(═O)NHCH₃, or —CH₃;

R² may be —H, —OH, —CH₃, -halo, —OC(═O)CH₃, —NHC(═O)CH₃, or —NO₂;

R³ may be —H, —OH, —CH₃, -halo, —OC(═O)CH₃, —NHC(═O)CH₃, or —NO₂;

R⁴ may be —H or —CH₃;

R⁵ may be —H, —OMe or —OH;

R⁶ may be —H, —OCH₃, or -halo;

R⁷ may be —H, —C(═O)CH₃, —CH₂C₆H₅, —CH₂—O—CH₂C₆H₅, —C(═O)O—C₆H₅OH, —C(═O)NHCH₃, or —CH₃;

R⁸ may be —H or —CH₃;

R⁹ may be —H or —CH₃; and

R¹⁰ may be —H or —CH₃.

In some embodiments, R¹, R², and R³ may each be —H.

In some embodiments, R⁴ may be —CH₃.

In some embodiments, R⁵ may be —OH. In some embodiments, R⁵ may be —H.

In some embodiments, R⁶ may be —H. In some embodiments, R⁶ may be —OCH₃.

In some embodiments, R⁷ may be —H.

In some embodiments, R⁸ may be —H. In some embodiments, R⁸ may be —CH₃.

In some embodiments, R⁹ may be —H. In some embodiments, R⁹ may be —CH₃.

In some embodiments, R¹⁰ may be —H. In some embodiments, R¹⁰ may be —CH₃.

In some embodiments described herein, the compound may selected from the group consisting of:

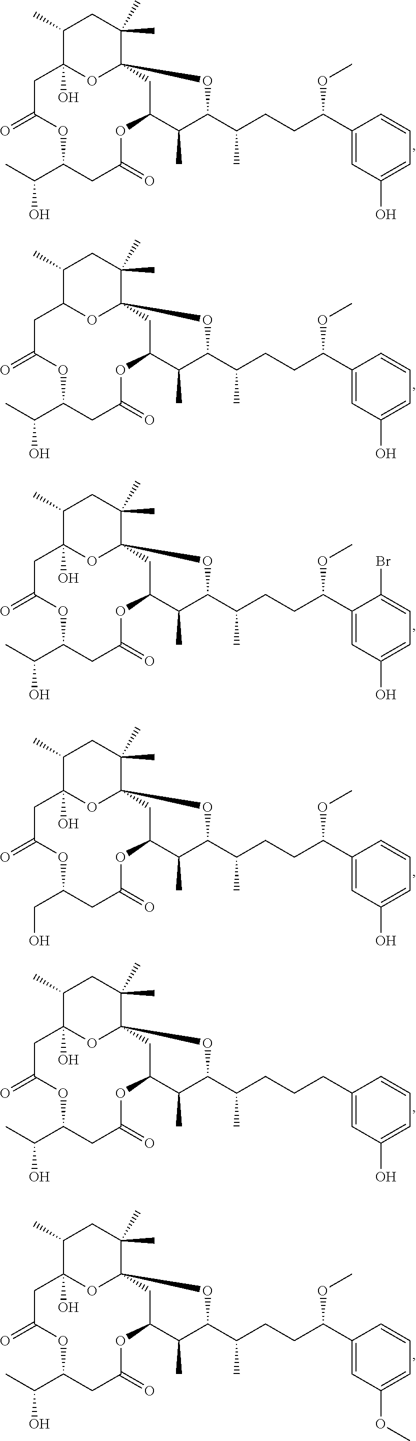

and pharmaceutically acceptable salts and prodrugs thereof.

Also disclosed herein is a method of reversing T-cell exhaustion in a subject, the method comprising administering to the subject a compound having the structure:

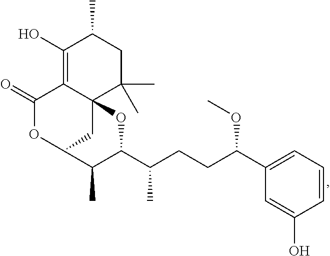 [SMILES: CO[C@@H](CC[C@H](C)[C@H]1O[C@@]23C[C@H](OC(=O)C2=C(O)[C@H](C)CC3(C)C)[C@@H]1C)c1cccc(O)c1]

or a pharmaceutically acceptable salt or prodrug thereof.

Also disclosed herein is a method of reversing T-cell exhaustion in a subject, the method comprising administering to the subject a compound of Formula (II):

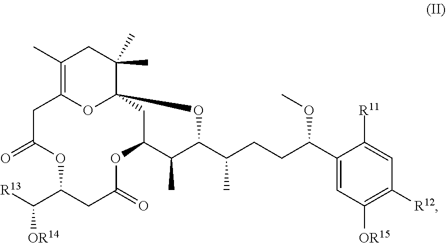

or a pharmaceutically acceptable salt or prodrug thereof, wherein:

R¹¹, R¹², and R¹³ may each independently be —H, halo, or —CH₃; and

R¹⁴ and R¹⁵ may each independently be —H, —C(═O)CH₃, —CH₂C₆H₅, —CH₂—O—CH₂C₆H₅, —C(═O)O—C₆H₅OH, —C(═O)NHCH₃, or —CH₃.

In some embodiments described herein, the compound may be

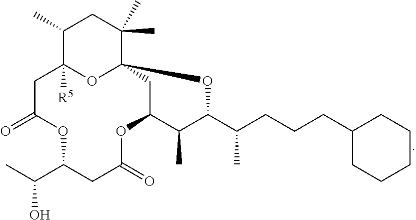

In some specific embodiments, R⁵ may be —H. In some specific embodiments, R⁵ may be —OH. In some specific embodiments, R⁵ may be —OMe.

In some embodiments described herein is a method of reversing T-cell exhaustion in a subject, the method comprising administering to the subject a Protein kinase C (PKC) theta activator.

In some embodiments, the PKC theta activator may be a phorbol ester. In some specific embodiments, the phorbol ester may be 12-O-tetradecanoylphorbol-13-acetate, deoxyphorbol-13-acetate (i.e., prostratin), or 12-deoxyphorbol-13-phenylacetate.

In some embodiments, the PKC theta activator may be a phorbol ester. In some specific embodiments, the phorbol ester may be 12-O-tetradecanoylphorbol-13-acetate, deoxyphorbol-13-acetate (i.e., prostratin), 12-deoxyphorbol-13-phenylacetate, prostratin, or phorbol-13 acetate.

In some embodiments, the PKC theta activator may be

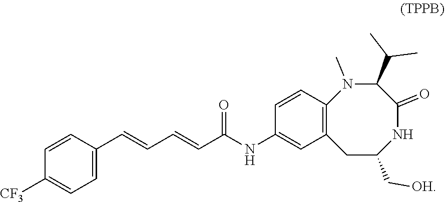 [SMILES: CC(C)[C@H]1C(=O)N[C@H](CO)Cc2cc(NC(=O)/C=C/C=C/c3ccc(C(F)(F)F)cc3)ccc2N1C]

In some embodiments, the PKC theta activator may be a teleocidin. In some specific embodiments, the teleocidin may be teleocidin A-1, teleocidin A-2, teleocidin B-1, teleocidin B-2, teleocidin B-3, teleocidin B-4, teleocidin B-18, des-O-methylolivoretin C, des-N-methylteleocidin B-4, blastmycetin A, blastnycetin B, blastmycetin C, blastmycetin D, blastmycetin E, blastmycetin F, (−)-indolactam-V, (−)-14-O-malonylindolactam-V, (−)-14-O-acetylindolactam-V, (−)-7-geranylindolactam-V, N13-desmethylteleocidin A-1, N13-desmethylteleocidin B-4, (−)-2-oxy-indolactam, olivoretin A (14-O-methylteleocidin B), olivoretin B, and olivoretin C, olivoretin A, olivoretin B, olivoretin C, olivoretin D, olivoretin E, des-O-methylolivoretin C, pendolmycin 14-O—(N-acetylglucosaminyl)teleocidin or (2E,4E)-N-((2S,5S)-5-(hydroxymethyl)-2-isopropyl-1-methyl-3-oxo-1,2,3,4,5,6-hexahydrobenzo[e][1,4]diazocin-8-yl)-5-(4-(trifluoromethyl)phenyl)penta-2,4-dienamide.

In some embodiments, the PKC theta activator may be ingenol or its ester derivatives. In some specific embodiments, the ingenol ester may be ingenol-3-angelate or ingenol-3-dodecanoate.

In some embodiments, the PKC theta activator may be farnesyl thiotriazole. In some embodiments, the PKC theta activator may be 2-[(2-pentylcyclopropyl)methyl]cyclopropaneoctanoic acid. In some embodiments, the PKC theta activator may be 5-chloro-N-(6-phenylhexyl)naphthalene-1-sulfonamide).

In some embodiments, the PKC theta activator may be a bryostatin. In some specific embodiments, the bryostatin may be bryostatin-1, bryostatin-2, brvostatin-3, bryostatin-4, bryostatin-5, bryostatin-6, bryostatin-7, bryostatin-8, or bryostatin-9.

In some embodiments, the compounds disclosed herein may be formulated into a pharmaceutical composition comprising one or pharmaceutically acceptable excipients.

In some embodiments, the methods disclosed herein may further comprise administering to the subject a second active agent. In some embodiments, the compound of Formula (I) or Formula (II), or any of the compounds disclosed herein and the second active agent may administered simultaneously. In some embodiments, the compound of Formula (I) or Formula (II), or any of the compounds disclosed herein and the second active agent may administered sequentially.

In some embodiments described herein, the subject may have cancer. In some embodiments, the cancer may be acute lymphoblastic leukemia, acute myeloid leukemia, bladder cancer, breast cancer, Burkitt lymphoma, cervical cancer, chronic lymphocytic leukemia, chronic myelogenous leukemia, colorectal cancer, esophageal cancer, Ewing sarcoma, gastric cancer, testicular cancer, renal cancer, hepatocellular cancer, melanoma, multiple myeloma, neuroblastoma, Hodgkin's lymphoma, non-Hodgkin lymphoma, pancreatic cancer, prostate cancer, rectal cancer, or thyroid cancer.

In some embodiments, the compounds described herein may be administered orally, intravenously, intraperitoneally, intragastrically, or intravascularly. In some embodiments, the composition is administered by intratumoral injection.

In another embodiment, provided herein is a method of reversing T-cell exhaustion in a subject, the method comprising the steps of: obtaining a biological sample comprising T-cells from the subject; contacting said T-cells with a compound of Formula (I) or Formula (II), or any of the compounds disclosed herein; and administering the T-cells contacted with said compound to said subject. In some embodiments, the method may further comprise administering a second active agent to the subject.

In yet another embodiment, provided herein is a method of shrinking a tumor in a subject, the method comprising administering to the subject a compound of Formula (I) or Formula (II), or any of the compounds disclosed herein. In some embodiments, the administration may be oral, intravenous, intraperitoneal, intragastric, or intravascular. In some embodiments, the composition may be administered by intratumoral injection. In some embodiments, administration of a compound of Formula (I) or Formula (II), or any of the compounds disclosed herein via intratumoral injection may result in the tumor shrinking by 10%, 20%, 30%, 50%, 70% or more.

In another embodiment, provided herein is a method of treating cancer comprising administering a compound of Formula (I) or Formula (II), or any of the compounds disclosed herein to a subject in need thereof by intratumoral injection.

In another embodiment provided herein is a method of improving the anti-tumor activity of a T cell, the method comprising: obtaining a biological sample comprising T-cells from the subject; contacting said T-cells with a compound of Formula (I) or Formula (II), or any of the compounds disclosed herein; and administering the T-cells contacted with said compound to said subject.

In another embodiment provided herein is a method of inducing a NFAT-dependent T cell activation in a subject, said method comprising administering a therapeutically effective amount of a pharmaceutical composition comprising a compound of Formula (I) or Formula (II), or any of the compounds disclosed herein to the subject. In some embodiments, the subject may have an infectious disease. In some embodiments, the subject may have a viral infection. In some embodiments, the viral infection may be HIV.

In another embodiment provided herein is a method of increasing the pool of immune checkpoint inhibitor responsive T cells in the tumor microenvironment.

In another embodiment provided herein is a method of increasing lymphocyte infiltration in the tumor microenvironment.

In another embodiment provided herein is a method of increasing the population of activated CD4+ and/or CD8+ cells in the tumor microenvironment.

### BRIEF DESCRIPTION OF THE DRAWINGS

FIG. 1 shows the activity Compound 1 as an NFAT promoter containing an IL-2 response element.

FIG. 2 shows the induction of CD69 expression in PMBCs after treatment with Compound 1.

FIG. 3 shows the effect of Compound 1 and anti-CD3 antibody on IL-2 production in PMBCs.

FIG. 4 shows the IFNγ secretion by cytomegalovirus (CMV)-specific T cells upon treatment with vehicle and Compound 1.

FIG. 5 shows the effect of a single intratumoral injection dose of Compound 1 on tumor size in a syngeneic melanoma mouse model.

FIG. 6 shows the reversal of T-cell exhaustion by ex vivo YFP expression.

FIG. 7 shows the effect of intratumoral injection of Compound 1 on lymphocyte infiltrate.

FIG. 8 shows the effect of intratumoral injection of Compound 1 on CD4+ CD69+ T cell infiltrate.

FIG. 9 shows the effect of intratumoral injection of Compound 1 on CD8+ CD69+ T cell infiltrate.

### DETAILED DESCRIPTION

### Definitions

The term “mammal” is used in its usual biological sense. Thus, it specifically includes humans and non-human mammals such as dogs, cats, horses, donkeys, mules, cows, domestic buffaloes, camels, llamas, alpacas, bison, yaks, goats, sheep, pigs, elk, deer, domestic antelopes, and non-human primates as well as many other species.

“Subject” as used herein, means a human or a non-human mammal including but not limited to a dog, cat, horse, donkey, mule, cow, domestic buffalo, camel, llama, alpaca, bison, yak, goat, sheep, pig, elk, deer, domestic antelope, or a non-human primate selected for treatment or therapy.

“Subject in need thereof” means a subject identified as in need of a therapy or treatment.

A therapeutic effect relieves, to some extent, one or more of the symptoms of a disease or disorder, and includes curing the disease or disorder. “Curing” means that the symptoms of active disease are eliminated. However, certain long-term or permanent effects of the disease may exist even after a cure is obtained (such as extensive tissue damage).

The phrase “therapeutically effective amount” means an amount of a compound or a combination of compounds that ameliorates, attenuates or eliminates one or more of the symptoms of a particular disease or condition or prevents, modifies, or delays the onset of one or more of the symptoms of a particular disease or condition.

“Treat,” “treatment,” or “treating,” as used herein refers to administering a pharmaceutical composition for prophylactic and/or therapeutic purposes. The term “prophylactic treatment” refers to treating a patient who does not yet have the relevant disease or disorder, but who is susceptible to, or otherwise at risk of, a particular disease or disorder, whereby the treatment reduces the likelihood that the patient will develop the disease or disorder. The term “therapeutic treatment” refers to administering treatment to a patient already having a disease or disorder.

“Preventing” or “prevention” refers to delaying or forestalling the onset, development or progression of a condition or disease for a period of time, including weeks, months, or years.

“Amelioration” means a lessening of severity of at least one indicator of a condition or disease. In certain embodiments, amelioration includes a delay or slowing in the progression of one or more indicators of a condition or disease. The severity of indicators may be determined by subjective or objective measures which are known to those skilled in the art.

“Modulation” means a perturbation of function or activity. In certain embodiments, modulation means an increase in gene expression. In certain embodiments, modulation means a decrease in gene expression. In certain embodiments, modulation means an increase or decrease in total serum levels of a specific protein. In certain embodiments, modulation means an increase or decrease in free serum levels of a specific protein. In certain embodiments, modulation means an increase or decrease in total serum levels of a specific non-protein factor. In certain embodiments, modulation means an increase or decrease in free serum levels of a specific non-protein factor. In certain embodiments, modulation means an increase or decrease in total bioavailability of a specific protein. In certain embodiments, modulation means an increase or decrease in total bioavailability of a specific non-protein factor.

“Administering” means providing a pharmaceutical agent or composition to a subject, and includes, but is not limited to, administering by a medical professional and self-administering.

Administration of the compounds disclosed herein or the pharmaceutically acceptable salts thereof, or the second pharmaceutical agents disclosed herein can be via any of the accepted modes of administration for agents that serve similar utilities including, but not limited to, orally, subcutaneously, intravenously, intranasally, topically, transdermally, intraperitoneally, intramuscularly, intrapulmonarilly, vaginally, rectally, or intraocularly. Oral and parenteral administrations are customary in treating the indications that are the subject of the preferred embodiments.

“Parenteral administration,” means administration through injection or infusion. Parenteral administration includes, but is not limited to, subcutaneous administration, intravenous administration, intramuscular administration, intraarterial administration, and intracranial administration.

“Subcutaneous administration” means administration just below the skin.

“Intravenous administration” means administration into a vein.

“Intraarterial administration” means administration into an artery.

“Intratumoral administration” means administration directly into a tumor.

The term “agent” includes any substance, molecule, element, compound, entity, or a combination thereof. It includes, but is not limited to, e.g., protein, polypeptide, peptide or mimetic, small organic molecule, polysaccharide, polynucleotide, and the like. It can be a natural product, a synthetic compound, or a chemical compound, or a combination of two or more substances.

“Pharmaceutical agent” means a substance that provides a therapeutic effect when administered to a subject.

“Pharmaceutical composition” means a mixture of substances suitable for administering to an individual that includes a pharmaceutical agent. For example, a pharmaceutical composition may comprise a modified oligonucleotide and a sterile aqueous solution.

“Active pharmaceutical ingredient” means the substance in a pharmaceutical composition that provides a desired effect.

The term “halogen” or “halo” refers to —F, —Cl, —Br and —I.

The term “pharmaceutically acceptable salt” refers to salts that retain the biological effectiveness and properties of the compounds with which they are associated and, which are not biologically or otherwise undesirable. In many cases, the compounds herein are capable of forming acid and/or base salts by virtue of the presence of phenol and/or phosphonate groups or groups similar thereto. One of ordinary skill in the art will be aware that the protonation state of any or all of these compounds may vary with pH and ionic character of the surrounding solution, and thus the present disclosure contemplates multiple charge states of each compound. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like; particularly preferred are the ammonium, potassium, sodium, calcium and magnesium salts. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. Many such salts are known in the art, as described in WO 87/05297, Johnston et al., published Sep. 11, 1987 (incorporated by reference herein in its entirety).

“Solvate” refers to the compound formed by the interaction of a solvent and an EPI, a metabolite, or salt thereof. Suitable solvates are pharmaceutically acceptable solvates including hydrates.

The term “prodrug” as used herein refers to any compound that when administered to a biological system generates a biologically active compound as a result of spontaneous chemical reaction(s), enzyme catalyzed chemical reaction(s), and/or metabolic chemical reaction(s), or a combination of each. Standard prodrugs are formed using groups attached to functionality, e.g., HO—, HS—, HOOC—, R₂N—, associated with the drug, that cleave in vivo. Standard prodrugs include but are not limited to carboxylate esters where the group is alkyl, aryl, aralkyl, acyloxyalkyl, alkoxycarbonyloxyalkyl as well as esters of hydroxyl, thiol and amines where the group attached is an acyl group, an alkoxycarbonyl, aminocarbonyl, phosphate or sulfate. The groups illustrated are exemplary, not exhaustive, and one skilled in the art could prepare other known varieties of prodrugs. Such prodrugs of the compounds of the present disclosure fall within this scope. Prodrugs must undergo some form of a chemical transformation to produce the compound that is biologically active or is a precursor of the biologically active compound. In some cases, the prodrug is biologically active, usually less than the drug itself, and serves to improve drug efficacy or safety through improved oral bioavailability, and/or pharmacodynamic half-life, etc. Prodrug forms of compounds may be utilized, for example, to improve bioavailability, improve subject acceptability such as by masking or reducing unpleasant characteristics such as bitter taste or gastrointestinal irritability, alter solubility such as for intravenous use, provide for prolonged or sustained release or delivery, improve ease of formulation, or provide site-specific delivery of the compound. Prodrugs are described in The Organic Chemistry of Drug Design and Drug Action, by Richard B. Silverman, Academic Press, San Diego, 1992. Chapter 8: “Prodrugs and Drug delivery Systems” pp. 352-401; Design of Prodrugs, edited by H. Bundgaard, Elsevier Science, Amsterdam, 1985; Design of Biopharmaceutical Properties through Prodrugs and Analogs, Ed. by E. B. Roche, American Pharmaceutical Association, Washington, 1977; and Drug Delivery Systems, ed. by R. L Juliano, Oxford Univ. Press, Oxford, 1980.

### Compounds

In some embodiments, the compounds for use as described herein include aplysiatoxin analogue compounds according to Formula I:

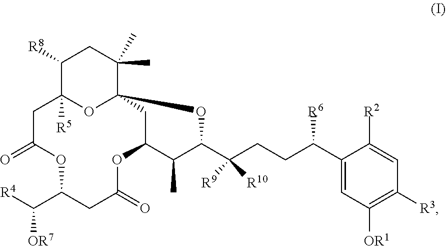

or a pharmaceutically acceptable salt or prodrug thereof, wherein:

R¹ is —H, —C(═O)CH₃, —CH₂C₆H₅, —CH₂—O—CH₂C₆H₅, C(═O)NHCH₃, or —CH₃;

R² is —H, —OH, —CH₃, -halo, —OC(═O)CH₃, —NHC(═O)CH₃, or —NO₂;

R³ is —H, —OH, —CH₃, -halo, —OC(═O)CH₃, —NHC(═O)CH₃, or —NO₂;

R⁴ is —H or —CH₃;

R⁵ is —H, —OMe or —OH;

R⁶ is —H, —OCH₃, or -halo;

R⁷ is —H, —C(═O)CH₃, —CH₂C₆H₅, —CH₂—O—CH₂C₆H₅, —C(═)O—C₆H₅OH, C(═O)NHCH₃, or —CH₃;

R⁸ is —H or —CH₃;

R⁹ is —H or —CH₃; and

R¹⁰ is —H or —CH₃.

In some embodiments, the compound is selected from one or more of the following:

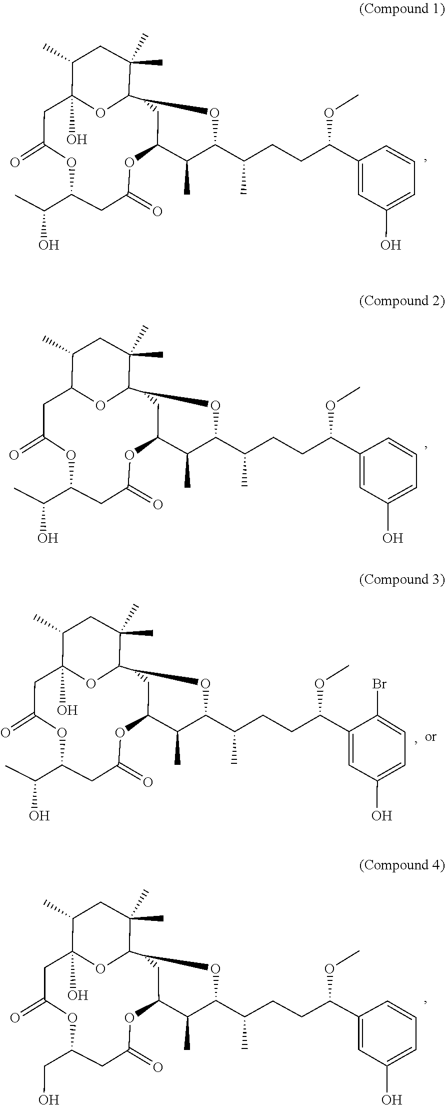

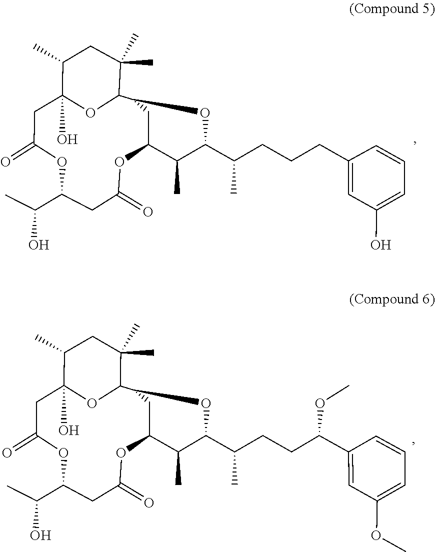

or pharmaceutically acceptable salts or prodrugs thereof.

In some embodiments, the compounds for use as described herein can be a compound having the structure:

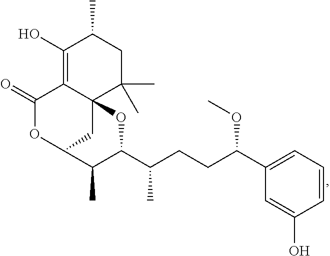 [SMILES: CO[C@@H](CC[C@H](C)[C@H]1O[C@@]23C[C@H](OC(=O)C2=C(O)[C@H](C)CC3(C)C)[C@@H]1C)c1cccc(O)c1]

or a pharmaceutically acceptable salt or prodrug thereof.

In some embodiments, the compounds for use as described herein include aplysiatoxin analogue compounds according to Formula (II):

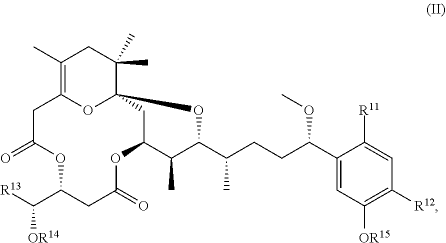

or a pharmaceutically acceptable salt or prodrug thereof, wherein:

R¹¹, R¹², and R¹³ are each independently —H, halo, or —CH₃; and

R¹⁴ and R¹⁵ are each independently —H, —C(═O)CH₃, —CH₂C₆H₅, —CH₂—O—CH₂C₆H₅, —C(═O)O—C₆H₅OH, —C(═O))NHCH₃, or —CH₃.

In some embodiments, the compound for use as described herein can be a compound having the structure:

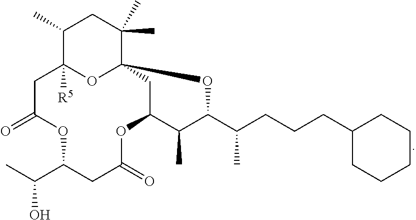

In some specific embodiments, R⁵ may be —H. In some specific embodiments, R⁵ may be —OH. In some specific embodiments, R⁵ may be —OMe.

The aplysiatoxin analogs described above may be isolated according to known methods, including those described in Scheuer et al., J. Am. Chem. Soc. 1974, 96(7):2245-2246; Mynderse et al., J. Org. Chem. 1978, 43(11):2301; and Nagai et al., J. Nat. Prod. 1997, 60, 925-928, each of which is incorporated herein by reference in its entirety. Synthesis of additional aplysiatoxin analogs is described in PCT Application Publication No. WO 2013/157555, which is incorporated herein by reference in its entirety.

In some embodiments, semisynthetic analogs of the aplysiatoxin analog compounds described herein can be prepared. For example, the compounds provided herein may be acetylated or alkylated according to the methods described by Kato et al, Pure Appl. Chem. 1975, 41, 1. In some embodiments, the compounds provided herein may be hydrogenated according to methods described in International Publication No. WO 2013/157555, Irie et al., Molecules 2017, 22, 631 and/or Motoyoshi et al, Tetrahedron 2006, 62, 1378, the entirety of each of which is incorporated by reference herein. Descriptions of the synthesis of aplysiotoxin analogs may be found in Kato, et al., Journal of the American Chemical Society 1974, 96(7), 2245-6; Mynderse, et al., Journal of Organic Chemistry 1978, 43(11), 2301-3. Moore, et al., Journal of Organic Chemistry 1984, 49(13), 2484-9. Park, et al,; Broka, Chris of the American Chemical Society 1987, 109(20), 6205-7; Walkup et al., Tetrahedron Letters 1987, 28(35), 4019-22; Ireland, et al., Journal of the American Chemical Society 1988, 110(17), 5768-79; Toshima, et al., Tetrahedron Letters 1989, 30(48), 6725-8; Okada, et al, Heterocycles 1991, 32(3), 431-6; Okamura, et al., Tetrahedron Letters 1991, 32(38), 5137-40; Okarnura, et al., Tetrahedron Letters 1991, 32(38), 5141-2; Okamura, et al., Tetrahedron (1993), 49(46), 10531-54; Nagai, et al; Journal of Natural Products (1997), 60(9), 925-928; Nagai, Hiroshi; Kan, Yukiko; Fujita, Tsuyoshi; Sakamoto, Bryan; Hokama, Yoshitsugi, Bioscience, Biotechnology, and Biochemistry 1998, 62(5), 1011-1013; Nakagawa, et al., Journal of the American Chemical Society 2009, 131(22), 7573-7579; Yanagita, et al., Bioorganic & Medicinal Chemistry Letters 2010, 20(20), 6064-6066; Nakagawa, et al., Bioscience, Biotechnology, and Biochemistry 2011, 75(6), 1167-1173; Shu, et al. Heterocycles 2012, 86(1), 281-303; Irie, et al., Pure and Applied Chemistry (2012), 84(6), 1341-1351; Kikumori, et al., Journal of Medicinal Chemistry (2012), 55(11), 5614-5626; Kamachi, et al., Bioorganic & Medicinal Chemistry (2013), 21(10), 2695-2702; Hanaki, et al., Tetrahedron (2013), 69(36), 7636-7645; Yanagita et al., Bioorganic & medicinal chemistry letters 2013, 23(15), 4319-23; Kikurnori, et al., Tetrahedron 2014, 70(52), 9776-9782; Gupta Deepak Kumar et al. Marine drugs 2014, 12(1), 115-27; Hanaki et al., Bioscience, biotechnology, and biochemistry 2015, 79(6), 888-95; Kikumori et al., Bioscience, Biotechnology, and Biochemistry 2016, 80(2), 221-231; Ashida, et al., Bioorganic & Medicinal Chemistry 2016, 24(18), 4218-4227; Hanaki, et al, Molecules (2017), 22(4), 631/1-631/13; Hanaki, et al., Biochemical and Biophysical Research Communications 2018, 495(1), 438-445, the entirety of each of which is incorporated by reference herein.

In some embodiments, the compounds for use as described herein include Protein kinase C (PKC) theta activators.

In some embodiments, the PKC theta activator may be a phorbol ester. In some specific embodiments, the phorbol ester may be 12-O-tetradecanoylphorbol-13-acetate or 12-deoxyphorbol-13-phenylacetate.

In some embodiments, the PKC theta activator may be a phorbol ester. In some specific embodiments, the phorbol ester may be 12-O-tetradecanoylphorbol-13-acetate, 12-deoxyphorbol-13-phenylacetate, prostratin, or phorbol-13 acetate.

In some embodiments, the PKC theta activator may be a teleocidin. In some specific embodiments, the teleocidin may be teleocidin A-1, teleocidin A-2, teleocidin B-1, teleocidin B-2, teleocidin B-3, teleocidin B-4, teleocidin B-18, des-O-methylolivoretin C, des-N-methylteleocidin B-4, blastmycetin A, blastnycetin B, blastmycetin C, blastmycetin D, blastmycetin E, blastmycetin F, (−)-indolactam-V, (−)-14-O-malonylindolactam-V, (−)-14-O-acetylindolactam-V, (−)-7-geranylindolactam-V, N13-desmethylteleocidin A-1, N13-desmethylteleocidin B-4, (−)-2-oxy-indolactam, olivoretin A (14-O-methylteleocidin B), olivoretin B, and olivoretin C, olivoretin A, olivoretin B, olivoretin C, olivoretin D, olivoretin E, des-O-methylolivoretin C, pendolmycin or 14-O—(N-acetylglucosaminyl)teleocidin, or (2E,4E)-N-((2S,5S)-5-(hydroxymethyl)-2-isopropyl-1-methyl-3-oxo-1,2,3,4,5,6-hexahydrobenzo[e][1,4]diazocin-8-yl)-5-(4-(trifluoromethyl)phenyl)penta-2,4-dienamide.

In some embodiments, the PKC theta activator may be

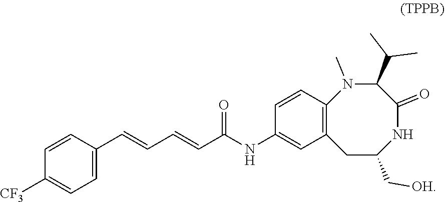 [SMILES: CC(C)[C@H]1C(=O)N[C@H](CO)Cc2cc(NC(=O)/C=C/C=C/c3ccc(C(F)(F)F)cc3)ccc2N1C]

In some embodiments, the PKC theta activator may be ingenol or its ester derivatives. In some specific embodiments, the ingenol ester may be ingenol-3-angelate or ingenol-3-dodecanoate.

In some embodiments, the PKC theta activator may be farnesyl thiotriazole. In some embodiments, the PKC theta activator may be 2-[(2-pentylcyclopropyl)methyl]cyclopropaneoctanoic acid. In some embodiments, the PKC theta activator may be 5-chloro-N-(6-phenylhexyl)naphthalene-1-sulfonamide)

In some embodiments, the PKC theta activator may be a bryostatin. In some specific embodiments, the bryostatin may be bryostatin-1, bryostatin-2, bryostatin-3, bryostatin-4, bryostatin-5, bryostatin-6, bryostatin-7, bryostatin-8, or bryostatin-9.

### Second Pharmaceutical Agents

The compounds presented herein may be administered in combination with one or more second pharmaceutical agents. In some embodiments, the compounds described above may be administered in combination with one second pharmaceutical agent. In some embodiments, the compounds described above may be administered in combination with two second pharmaceutical agents. In some embodiments, the compounds described above may be administered in combination with three or more second pharmaceutical agents.

In some embodiments, the compounds presented herein may be administered simultaneously with one or more second pharmaceutical agents. In other embodiments, the compounds of the present disclosure may be administered sequentially with one or more second pharmaceutical agents.

In some embodiments, the second pharmaceutical agent may be a chemotherapeutic agent selected from, but not limited to, an alkyating agent (e.g., cisplatin, carboplatin, mechlorethamine, cyclophosphamide, chlorambucil, ifosfamide and/or oxaliplatin) an anti-metabolite (e.g., azathioprine and/or mercaptopurine); a terpenoid (e.g., a vinca alkaloid and/or a taxane; e.g., Vincristine, Vinblastine, Vinorelbine and/or Vindesine, Taxo, Paclitaxel and/or Docetaxel); a topoisomerase (e.g., a type I topoisomerase and/or a type 2 topoisomnerase; e.g., camptothecins, such as irinotecan and/or topotecan; amsacrine, etoposide, etoposide phosphate and/or teniposide); a cytotoxic antibiotic (e.g., actinomycin, anthracyclines, doxorubicin, daunorubicin, valrubicin, idarubicin, epirubicin, bleomycin, plicamycin and/or mitomycin); a hormone (e.g., a lutenizing hormone releasing hormone agonist; e.g., leuprolidine, goserelin, triptorelin, histrelin, bicalutamide, flutamide and/or nilutamide); an antibody (e.g., Abciximab, Adalimumab, Alemtuzumab, Atlizumab, Basiliximab, Belimumab, Bevacizumab, Bretuximab vedotin, Canakinumab, Cetuximab, Ceertolizumab pegol, Daclizumab, Denosumab, Eculizumab, Efalizumab, Gemtuzumab, Golimumab, Golimumab, Ibritumomab tiuxetan, Infliximab, Ipilimumab, Muromonab-CD3, Natalizumab, Ofatumumab, Omalizumab, Palivizumab, Panitumumab, Ranibizumab, Rituximab, Tocilizumab, Tositumomab and/or Trastuzumab): an anti-angiogenic agent; a cytokine; a thrombotic agent; a growth inhibitory agent; an anti-helminthic agent; and an immune checkpoint inhibitor that targets an immune checkpoint receptor selected from the group consisting of CTLA-4, PD-1, PD-L1, PD-1-PD-L1, PD-1-PD-L2, interleukin-2 (IL-2), indoleamine 2,3-dioxygenase (IDO), IL-10, transforming growth factor-β (TGFβ), T cell immunoglobulin and mucin 3 (TIM3 or HAVCR2), Galectin 9-TIM3 Phosphatidylserine-TIM3 lymphocyte activation gene 3 protein (LAG3), MHC class II-LAG3, 4-1BB-4-1BB ligand, OX40-OX40 ligand, GITR, GITR ligand-GITR, CD27, CD70-CD27, TNFRSF25, TNFRSF25-TL1A, CD40L, CD40-CD40 ligand, HVEM-LIGHT-LTA, HVEM, HVEM-BTLA, HVEM-CD160, HVEM-LIGHT, HVEM-BTLA-CD160, CD80, CD80-PDL-1, PDL2-CD80, CD244, CD48-CD244, ICOS, ICOS-ICOS ligand, B7-H3, B7-H4, VISTA, TMIGD2, HHLA2-TMIGD2, Butyrophilins, including BTNL2, Siglec family, TIGIT and PVR family members, KIRs, ILTs and LIRs, NKG2D and NKG2A, MICA and MICB, CD244, CD28, CD86-CD28, CD86-CTLA, CD80-CD28, CD39, CD73 Adenosine-CD39-CD73, CXCR4-CXCL12, Phosphatidylserine, TIM3, Phosphatidylserine-TIM3, SIRPA-CD47, VEGF, Neuropilin, CD160, CD30, and CD155 (e.g., CTLA-4 or PD1 or PD-L1).

### Pharmaceutical Compositions

The compounds as described above and/or the second pharmaceutical agents described above can be formulated into pharmaceutical compositions for use in treatment of the conditions described herein Standard pharmaceutical formulation techniques are used, such as those disclosed in Remington's The Science and Practice of Pharmacy, 21st Ed., Lippincott Williams & Wilkins (2005), incorporated herein by reference in its entirety. Accordingly, some embodiments include pharmaceutical compositions comprising: (a) a safe and therapeutically effective amount of a compound described herein, or pharmaceutically acceptable salts thereof; and (h a pharmaceutically acceptable carrier, diluent, excipient or combination thereof.

In some embodiments, the compounds provided herein and the second pharmaceutical agents provided herein may be formulated into a single pharmaceutical composition for use in treatment of the conditions described herein. In some embodiments, a formulation comprising the compounds provided herein may be administered in combination with one or more second pharmaceutical agents provided herein or a pharmaceutical composition comprising one or more second pharmaceutical agents provided herein.

The term “pharmaceutically acceptable carrier” or “pharmaceutically acceptable excipient” includes any and all solvents, diluents, emulsifiers, binders, buffers, dispersion media, coatings, antibacterial and antifungal agents, isotonic and absorption delaying agents and the like, or any other such compound as is known by those of skill in the art to be useful in preparing pharmaceutical formulations. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions. In addition, various adjuvants such as are commonly used in the art may be included. These and other such compounds are described in the literature, e.g., in the Merck Index, Merck & Company, Rahway, N.J. Considerations for the inclusion of various components in pharmaceutical compositions are described, e.g., in Gilman et al. (Eds.) (1990); Goodman and Gilman's: The Pharmacological Basis of Therapeutics, 8th Ed. Pergamon Press.

Some examples of substances, which can serve as pharmaceutically-acceptable carriers or components thereof, are sugars, such as lactose, glucose and sucrose; starches, such as corn starch and potato starch; cellulose and its derivatives, such as sodium carboxymethyl cellulose, ethyl cellulose, and methyl cellulose; powdered tragacanth; malt; gelatin; talc; solid lubricants, such as stearic acid and magnesium stearate; calcium sulfate; vegetable oils, such as peanut oil, cottonseed oil, sesame oil, olive oil, corn oil and oil of theobroma; polyols such as propylene glycol, glycerine, sorbitol, mannitol, and polyethylene glycol; alginic acid; emulsifiers, such as the TWEENS; wetting agents, such as sodium lauryl sulfate; coloring agents; flavoring agents; tableting agents, stabilizers; antioxidants; preservatives; pyrogen-free water; isotonic saline; and phosphate buffer solutions.

The choice of a pharmaceutically-acceptable carrier to be used in conjunction with the subject compound is determined by the way the compound is to be administered.

The compositions described herein are preferably provided in unit dosage form. As used herein, a “unit dosage form” is a composition containing an amount of a compound that is suitable for administration to a subject, in a single dose, according to good medical practice. The preparation of a single or unit dosage form however, does not imply that the dosage form is administered once per day or once per course of therapy. A unit dosage form may comprise a single daily dose or a fractional sub-dose wherein several unit dosage forms are to be administered over the course of a day in order to complete a daily dose. According to the present disclosure, a unit dosage form may be given more or less often that once daily, and may be administered more than once during a course of therapy. Such dosage forms may be administered in any manner consistent with their formulation, including orally, parenterally, and may be administered as an infusion over a period of time (e.g., from about 30 minutes to about 2-6 hours). While single administrations are specifically contemplated, the compositions administered according to the methods described herein may also be administered as a continuous infusion or via an implantable infusion pump.

The methods as described herein may utilize any of a variety of suitable forms for a variety of routes for administration, for example, for oral, nasal, rectal, topical (including transdermal), ocular, intracerebral, intracranial, intrathecal, intra-arterial, intravenous, intratumoral, intramuscular, or other parental routes of administration. The skilled artisan will appreciate that oral and nasal compositions include compositions that are administered by inhalation, and made using available methodologies. Depending upon the particular route of administration desired, a variety of pharmaceutically-acceptable carriers well-known in the art may be used. Pharmaceutically-acceptable carriers include, for example, solid or liquid fillers, diluents, hydrotropes, surface-active agents, and encapsulating substances. Optional pharmaceutically-active materials may be included, which do not substantially interfere with the activity of the compound. The amount of carrier employed in conjunction with the compound is sufficient to provide a practical quantity of material for administration per unit dose of the compound. Techniques and compositions for making dosage forms useful in the methods described herein are described in the following references, all incorporated by reference herein: Modern Pharmaceutics, 4th Ed., Chapters 9 and 10 (Banker & Rhodes, editors, 2002); Lieberman et al., Pharmaceutical Dosage Forms: Tablets (1989); and Ansel, Introduction to Pharmaceutical Dosage Forms 8th Edition (2004).

Various oral dosage forms can be used, including such solid forms as tablets, capsules, granules and bulk powders. Tablets can be compressed, tablet triturates, enteric-coated, sugar-coated, film-coated, or multiple-compressed, containing suitable binders, lubricants, diluents, disintegrating agents, coloring agents, flavoring agents, flow-inducing agents, and melting agents. Liquid oral dosage forms include aqueous solutions, emulsions, suspensions, solutions and/or suspensions reconstituted from non-effervescent granules, and effervescent preparations reconstituted from effervescent granules, containing suitable solvents, preservatives, emulsifying agents, suspending agents, diluents, sweeteners, melting agents, coloring agents and flavoring agents.

The pharmaceutically-acceptable carriers suitable for the preparation of unit dosage forms for peroral administration is well-known in the art. Tablets typically comprise conventional pharmaceutically-compatible adjuvants as inert diluents, such as calcium carbonate, sodium carbonate, mannitol, lactose and cellulose; binders such as starch, gelatin and sucrose; disintegrants such as starch, alginic acid and croscarmelose; lubricants such as magnesium stearate, stearic acid, microcrystalline cellulose, carboxymethyl cellulose, and talc. Tablets may also comprise solubilizers or emulsifiers, such as poloxamers, cremophor/Kolliphor®/Lutrol®, methylcellulose, hydroxypropylmethylcellulose, or others as are known in the art. Glidants such as silicon dioxide can be used to improve flow characteristics of the powder mixture. Coloring agents, such as the FD&C dyes, can be added for appearance. Sweeteners and flavoring agents, such as aspartame, saccharin, menthol, peppermint, and fruit flavors, are useful adjuvants for chewable tablets. Capsules typically comprise one or more solid diluents disclosed above. The selection of carrier components depends on secondary considerations like taste, cost, and shelf stability, which can be readily made by a person skilled in the art.

Peroral (PO) compositions also include liquid solutions, emulsions, suspensions, and the like. The pharmaceutically-acceptable carriers suitable for preparation of such compositions are well known in the art. Typical components of carriers for syrups, elixirs, emulsions and suspensions include ethanol, glycerol, propylene glycol, polyethylene glycol, liquid sucrose, sorbitol and water. For a suspension, typical suspending agents include methyl cellulose, sodium carboxymethyl cellulose, AVICEL RC-591, tragacanth and sodium alginate; typical wetting agents include lecithin and polysorbate 80; and typical preservatives include methyl paraben and sodium benzoate. Peroral liquid compositions may also contain one or more components such as sweeteners, flavoring agents and colorants disclosed above.

Such compositions may also be coated by conventional methods, typically with pH or time-dependent coatings, such that the subject compound is released in the gastrointestinal tract in the vicinity of the desired topical application, or at various times to extend the desired action. Such dosage forms typically include, but are not limited to, one or more of cellulose acetate phthalate, polyvinylacetate phthalate, hydroxypropyl methyl cellulose phthalate, ethyl cellulose, Eudragit coatings, waxes and shellac.

Compositions described herein may optionally include other drug actives.

Other compositions useful for attaining systemic delivery of the subject compounds include sublingual, buccal and nasal dosage forms. Such compositions typically comprise one or more of soluble filler substances such as sucrose, sorbitol and mannitol; and binders such as acacia, microcrystalline cellulose, carboxymethyl cellulose and hydroxypropyl methyl cellulose. Glidants, lubricants, sweeteners, colorants, antioxidants and flavoring agents disclosed above may also be included.

A liquid composition, which is formulated for topical ophthalmic use, is formulated such that it can be administered topically to the eye. The comfort may be maximized as much as possible, although sometimes formulation considerations (e.g. drug stability) may necessitate less than optimal comfort. In the case that comfort cannot be maximized, the liquid may be formulated such that the liquid is tolerable to the patient for topical ophthalmic use. Additionally, an ophthalmically acceptable liquid may either be packaged for single use, or contain a preservative to prevent contamination over multiple uses.

For ophthalmic application, solutions or medicaments are often prepared using a physiological saline solution as a major vehicle. Ophthalmic solutions may preferably be maintained at a comfortable pH with an appropriate buffer system. The formulations may also contain conventional, pharmaceutically acceptable preservatives, stabilizers and surfactants.

Preservatives that may be used in the pharmaceutical compositions disclosed herein include, but are not limited to, benzalkonium chloride, PHMB, chlorobutanol, thimerosal, phenylmercuric, acetate and phenylmercuric nitrate. A useful surfactant is, for example, Tween 80. Likewise, various useful vehicles may be used in the ophthalmic preparations disclosed herein. These vehicles include, but are not limited to, polyvinyl alcohol, povidone, hydroxypropyl methyl cellulose, poloxamers, carboxymethyl cellulose, hydroxyethyl cellulose and purified water.

Tonicity adjustors may be added as needed or convenient. They include, but are not limited to, salts, particularly sodium chloride, potassium chloride, mannitol and glycerin, or any other suitable ophthalmically acceptable tonicity adjustor.

Various buffers and means for adjusting pH may be used so long as the resulting preparation is ophthalmically acceptable. For many compositions, the pH will be between 4 and 9. Accordingly, buffers include acetate buffers, citrate buffers, phosphate buffers and borate buffers. Acids or bases may be used to adjust the pH of these formulations as needed.

Ophthalmically acceptable antioxidants include, but are not limited to, sodium metabisulfite, sodium thiosulfate, acetylcysteine, butylated hydroxyanisole and butylated hydroxytoluene.

Other excipient components, which may be included in the ophthalmic preparations, are chelating agents. A useful chelating agent is edetate disodium, although other chelating agents may also be used in place or in conjunction with it.

For topical use, including for transdermal administration, creams, ointments, gels, solutions or suspensions, etc., containing the compound disclosed herein are employed. Topical formulations may generally be comprised of a pharmaceutical carrier, co-solvent, emulsifier, penetration enhancer, preservative system, and emollient.

For intravenous administration, the compounds and compositions described herein may be dissolved or dispersed in a pharmaceutically acceptable diluent, such as a saline or dextrose solution. Suitable excipients may be included to achieve the desired pH, including but not limited to NaOH, sodium carbonate, sodium acetate, HCl, and citric acid. In various embodiments, the pH of the final composition ranges from 2 to 8, or preferably from 4 to 7. Antioxidant excipients may include sodium bisulfite, acetone sodium bisulfite, sodium formaldehyde, sulfoxylate, thiourea, and EDTA. Other non-limiting examples of suitable excipients found in the final intravenous composition may include sodium or potassium phosphates, citric acid, tartaric acid, gelatin, and carbohydrates such as dextrose, mannitol, and dextran. Further acceptable excipients are described in Powell, et al., Compendium of Excipients for Parenteral Formulations, PDA J Pharm Sci and Tech 1998, 52 238-311 and Nema et al., Excipients and Their Role in Approved Injectable Products: Current Usage and Future Directions, PDA J. Pharm. Sci. Tech. 2011, 65 287-332, both of which are incorporated herein by reference in their entirety. Antimicrobial agents may also be included to achieve a bacteriostatic or fungistatic solution, including but not limited to phenylmercuric nitrate, thimerosal, benzethonium chloride, benzalkonium chloride, phenol, cresol, and chlorobutanol.

The compositions for intravenous administration may be provided to caregivers in the form of one more solids that are reconstituted with a suitable diluent such as sterile water, saline or dextrose in water shortly prior to administration. In other embodiments, the compositions are provided in solution ready to administer parenterally. In still other embodiments, the compositions are provided in a solution that is further diluted prior to administration. In embodiments that include administering a combination of a compound described herein and another agent, the combination may be provided to caregivers as a mixture, or the caregivers may mix the two agents prior to administration, or the two agents may be administered separately.

The actual unit dose of the compounds described herein and/or second pharmaceutical agents described herein depends on the specific compound, and on the condition to be treated. In some embodiments, the dose may be from about 0.01 mg/kg to about 120 mg/kg or more of body weight, from about 0.05 mg/kg or less to about 70 mg/kg, from about 0.1 mg/kg to about 50 mg/kg of body weight, from about 1.0 mg/kg to about 10 mg/kg of body weight, from about 5.0 mg/kg to about 10 mg/kg of body weight, or from about 10.0 mg/kg to about 20.0 mg/kg of body weight. In some embodiments, the dose may be less than 100 mg/kg, 90 mg/kg, 80 mg/kg, 70 mg/kg, 60 mg/kg, 50 mg/kg, 40 mg/kg, 30 mg/kg, 25 mg/kg, 20 mg/kg, 10 mg/kg, 7.5 mg/kg, 6 mg/kg, 5 mg/kg, 4 mg/kg, 3 mg/kg, 2.5 mg/kg, 1 mg/kg, 0.5 mg/kg, 0.1 mg/kg, 0.05 mg/kg or 0.005 mg/kg of body weight. In some embodiments, the actual unit dose is 0.05, 0.07, 0.1, 0.3, 1.0, 3.0, 5.0, 10.0 or 25.0 mg/kg of body weight. Thus, for administration to a 70 kg person, the dosage range would be from about 0.1 mg to 70 mg, from about 1 mg to about 50 mg, from about 0.5 mg to about 10 mg, from about 1 mg to about 10 mg, from about 2.5 mg to about 30 mg, from about 35 mg or less to about 700 mg or more, from about 7 mg to about 600 mg, from about 10 mg to about 500 mg, or from about 20 mg to about 300 mg, or from about 200 mg to about 2000 mg. In some embodiments, the actual unit dose is 5 mg. In some embodiments the actual unit dose is 10 mg. In some embodiments, the actual unit dose is 25 mg. In some embodiments, the actual unit dose is 250 mg or less. In some embodiments, the actual unit dose is 100 rg or less. In some embodiments, the actual unit dose is 70 mg or less.

The compounds described herein and/or the second pharmaceutical agents described herein may also be incorporated into formulations for delivery outside the systemic circulation. Such formulations may include enteric-coated capsules, tablets, soft-gels, spray dried powders, polymer matrices, hydrogels, enteric-coated solids, crystalline solids, amorphous solids, glassy solids, coated micronized particles, liquids, nebulized liquids, aerosols, or microcapsules.

### Methods of Administration

The compositions described above may be administered through any suitable route of administration, for example, by injection, such as subcutaneously, intramuscularly, intraperitoneally, intratumorally, intravenously, or intraarterially; topically, such as by cream, lotion, or patch; orally, such as by a pill, dissolved liquid, oral suspension, buccal film, or mouth rinse; nasally, such as by a nasal aerosol, powder, or spray; or ocularly, such as by an eye drop). In some embodiments, the composition may be administered one, twice, three times, our four times per day. In other embodiments, the composition may be administered once, twice, or three times per week. In other embodiments, the composition is administered every other day, every three days, or every four days. In other embodiments, the composition every other week, every three weeks, or every four weeks. In other embodiments, the composition is administered once per month or twice per month.

In some embodiments, an initial loading dose is administered which is higher than subsequent doses (maintenance doses). The dosage form or mode of administration of a maintenance dose may be different from that used for the loading dose. In any of the embodiments disclosed herein, a maintenance dose may comprise administration of the unit dosage form on any dosing schedule contemplated herein, including but not limited to, monthly or multiple times per month, biweekly or multiple times each two weeks, weekly or multiple times per week, daily or multiple times per day. It is contemplated within the present disclosure that dosing holidays may be incorporated into the dosing period of the maintenance dose. Such dosing holidays may occur immediately after the administration of the loading dose or at any time during the period of administration of the maintenance dose. In some embodiments, the loading dose is 300 mg or less; 250 mg or less, 200 mg or less, 150 mg or less, or 100 mg or less. In some embodiments, the maintenance dose is 300 mg or less; 200 mg or less, 100 mg or less, 50 mg or less, 25 mg or less, 10 mg or less, 5 mg or less, or 1 mg or less.

In some embodiments, the compounds presented herein may be administered simultaneously with one or more second pharmaceutical agents. In other embodiments, the compounds of the present disclosure may be administered sequentially with one or more second pharmaceutical agents.

In some embodiments, the compounds may be administered prior to administration of the second pharmaceutical agent. In some embodiments the compounds may be administered about 15 minutes, about 30 minutes, about 45 minutes, about 1 hour, about 2 hours, about 3 hours, about 4 hours, about 6 hours, about 8 hours, about 12 hours, or about 24 hours prior to administration of a second pharmaceutical agent provided herein. In some embodiments, the compounds may be administered after administration of the second pharmaceutical agent. In some embodiments the compounds may be administered about 15 minutes, about 30 minutes, about 45 minutes, about 1 hour, about 2 hours, about 3 hours, about 4 hours, about 6 hours, about 8 hours, about 12 hours, or about 24 hours after administration of a second pharmaceutical agent provided herein.

In some embodiments, T cells treated with the compounds or compositions disclosed herein may be administered to as subject. For example, in some embodiments, such administration may include the steps of (i) obtaining a biological sample comprising T-cells from the subject; (ii) contacting said T-cells with a compound or composition disclosed herein; and (iii) administering the T-cells contacted with said compounds or compositions to said subject.

### Methods of Treatment

Some embodiments according to the methods and compounds or compositions of the present disclosure relate to a method for reversing T cell exhaustion in subject. In some embodiments, the methods and compounds or compositions of the present disclosure relate to a method for preventing, treating, or ameliorating cancer. In some embodiments, the cancer may be acute lymphoblastic leukemia, acute myeloid leukemia, bladder cancer, breast cancer, Burkitt lymphoma, cervical cancer, chronic lymphocytic leukemia, chronic myelogenous leukemia, colorectal cancer, esophageal cancer, Ewing sarcoma, gastric cancer, testicular cancer, renal cancer, hepatocellular cancer, melanoma, multiple myeloma, neuroblastoma, Hodgkin's lymphoma, non-Hodgkin lymphoma, pancreatic cancer, prostate cancer, rectal cancer, or thyroid cancer.

In some embodiments according to the methods and compounds or compositions of the present disclosure relate to a method for shrinking a tumor in a subject. In some embodiments, the tumor may shrink by 10% as compared to the size of the tumor immediately prior to administration of the compositions disclosed herein to a subject. In some embodiments, the tumor may shrink by 15%, 20%, 25%, 30%, 35%, 40%, 45%, 50%, 55%, 60%, 65%, 70%, 75%, 80%, 85%, 90%, 95%, or 100% as compared to the size of the tumor immediately prior to administration of the compositions disclosed herein to a subject.

In some embodiments according to the methods and compounds or compositions of the present disclosure relate to a method for inducing a NFAT-dependent T cell activation in a subject.

In some embodiments, the subject may have an infectious disease. In some embodiments, the subject may have a viral infection.

The methods described herein are further illustrated by the following examples.

### Example 1: Isolation of Compound 1 and Aplysiatoxin

Compound 1 was isolated according to procedures described in Moore, R. E. et al., J. Org. Chem. 1984, 49, 2484. An unidentified macroalgae and cyanobacteria mixture (SBM420) was collected at an intertidal zone from Molokai, Hi., (Latitude 21.08677, Longitude (−157.251) on Feb. 7, 2018. Specimens were preserved by freezing directly and stored at −20° C. until extraction.

A frozen mixture of macroalgae and cyanobacteria (SBM420; 661 g) were thawed and exhaustively extracted with methanol and methanol/dichloromethane (1:1) respectively. The organic layers were combined, concentrated under reduced pressure to yield a greenish crude extract (9.23 g). The crude extract was then subjected to a solid phase extraction (SPE) by loading on a HP20 resin using Combiflash® Rf+ system. The resin was then washed stepwise with 100% water, water/isopropanol (1:1), water/isopropanol (1:2), methanol, and dichloromethane to generating 5 fractions. The fraction 2-3 were combined and concentrated under reduced pressure to yield 1.95 g of SPE extract. A portion of SPE extract (1.45 g) was then fractionated using reversed-phase flash chromatography (50 g C18 Gold column) on Combiflash® Rf+ system with water/methanol gradient (0-100% Methanol over 30 minutes) to yield 98 fractions. All fractions were analyzed by LCMS to identify the fractions containing Compound 1 and aplysiatoxin.

The fractions that contained Compound 1 (fractions 46-50) were combined, concentrated under reduced pressure (219 mg) and repeated the same flash chromatography using water/acetonitrile gradient to yield a semi-pure sample of Compound 1 (89 mg). The semi-pure sample was further purified by preparative reversed-phase HPLC (Luna C18(2), 150×30 mm, 5 μm; 25 mL min⁻¹, isocratic 60% aqueous acetonitrile over 25 min). Compound 1 was collected as 4 fractions eluting between 13.5 and 15.5 min. Fractions containing Compound 1 (as determined by LCMS) were pooled and concentrated in vacuo to yield 53.1 mg of pure Compound 1 as determined LCMS and ¹H-NMR.¹

The fractions that contained aplysiatoxin (fractions 53-55) were combined, concentrated under reduced pressure (174 mg) and repeated the flash chromatography using different gradient system (0-100% Acetonitrile in water over 30 minutes) to yield a semi-pure sample of aplysiatoxin (69 mg; ˜505 pure). The semi-pure sample was further purified by preparative reversed-phase HPLC (Luna C18(2), 150×30 mm, 5 μm; 25 mL min⁻¹, a narrow gradient 50-100% aqueous Acetonitrile over 30 min). Fractions containing aplysiatoxin (as determined by LCMS) were pooled and concentrated in vacuo to yield a pure sample of aplysiatoxin (254 mg). The compound identity was confirmed by LCMS and 1H-NMR.¹

### Example 2: NFAT Activity Assay

The nuclear factor of activator T cells (NFAT) family of transcription factors plays an important role in immune response. T cell activation through the T cell synapse results in calcium influx. Increased intracellular calcium levels activate the calcium-sensitive phosphatase, calcineurin, which rapidly dephosphorylates the serine-rich region (SRR) and SP-repeats in the amino termini of NEAT proteins. This results in a conformational change that exposes a nuclear localization signal promoting NFAT nuclear import. In the nucleus, NFAT proteins cooperate with other proteins to bind to DNA.

The NFAT assay was used to determine the activity of Compound 1. Jurkat-NFAT-luc cells (BPS #60621) and One-step Luciferase assay system (BPS #60690-1) were purchased from BPS bioscience. Concanavalin A/ConA was obtained from Sigma (Sigma #C5275, 5 mg/ml in PBS), as was Ionomycin (Sigma #I3909-1 ml, 1 mM in DMSO).

Preparation of Intermedia dilution plates (1:20) with media: Complete media (28.5 μL) was manually pipetted into intermedia dilution plates. 1.5 μL of compound solution from compound plates were added into intermedia dilution plates and mixed well.

Preparation of cell plates (1:10 with cell solution): Jurkat-NFAT-luc cells were collected and counted. A cell solution was prepared by combining 5×10⁶ cells in 11.25 mL media with 8.4 μL of Ionomycin (stock solution: 1 mM, working solution: 0.75 μM) (for one 384-well plate). 22.5 μL of the above cell solution was manually pipetted into 384-well white plate. 2.5 μL of compound solution was manually transferred from intermedia dilution plates into 384-well cell plates. 2.5 μL of HPC (ConA: 15 μg/mL) was pipetted into one column of wells, while 2.5 μL of LPC (ConA: 5 μg/mL) into a second column. The cells were incubated at 37° C. for 18 hrs. 18 uL of one step luciferase detection reagent was then added to each well and the plates were read.

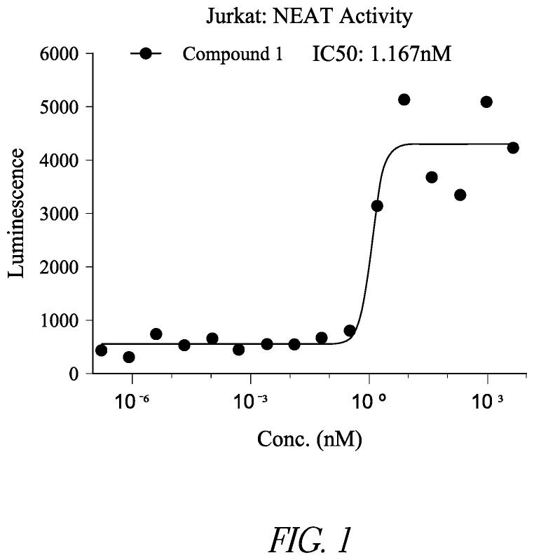

FIG. 1 shows the NFAT activity of Compound 1 with an IC₅₀ of 1.167 nM. Compound 1 activates the NFAT promoter containing an IL-2 response element. This activation is dependent on the presence of ionomycin, and demonstrates a role for calcium mobilization in the activity of Compound 1.

### Example 3: IL-2 Production Assay

This assay was performed to evaluate the IC₅₀ of IL-2 production with Compound 1 plus ionomycin, anti-CD3 (aCD3), or anti-CD-28 (aCD28). The following materials were used for the assay: Peripheral blood mononuclear cells (“PBMC”s) (iXCells #50-107-7995, 25×106, Lot #200127); Ionomycin (Sigma #I3909-1 mL, 1 mM in DMSO); CD3 (ebioscience #16-0037-85, 1 mg/mL); CD28 (ebioscience #16-0289-85, 1 mg/mL); and IL-2 ELISA (BD #5506111). PBMCs were thawed with AIM-V media one day before assay.

A column in the 96-well plate was coated with 0.04 μg/mL aCD3 at 37° C. for 0.5 hr (0.5 μL aCD3 (1 mg/mL)+12.5 mL AIM-V). The solution was removed from the plate and the plate was then washed with PBS solution three times. A 7.5 μM solution of lonomycin was prepared by combining 1.5 μL lonomycin (1 mM)+200 μL AIM-V. Similarly, a 2.5 μg/mL solution of CD28 was prepared by combining 0.5 μL aCD28 (1 mg/mL)+200 μL AIM-V. Additionally, a 50 μM solution of Compound 1 was prepared in AIM-V. Serial dilution of the stock solution were performed by combining 45 μL AIM-V with 2.5 μL Compound 1. A control solution of 5% DMSO was prepared in AIM-V media. 40 μL of AIM-V media and 50 μL of the above cell solution into desired wells. 10 μL of either Compound 1 solutions or controls were added into the desired wells. The plate was incubated at 37° C. for 24 hrs. The next day, everything was transferred into a V-bottom 96 well plate and spun at 300 g for 5 min. The supernatant was then transferred into a new 96-well plate as ELISA samples and measured for soluble IL-2.

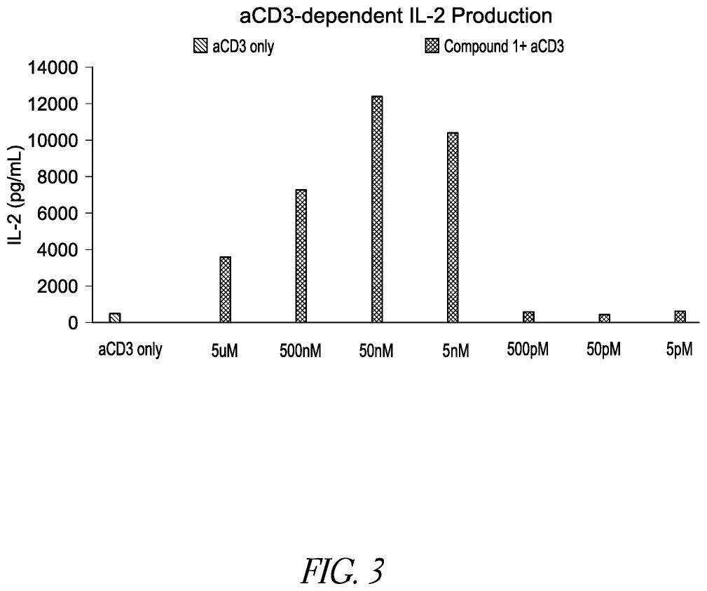

The results of this dose-dependent IL-2 production shows that expression of IL-2 is induced in PMBCs by treatment with Compound 1 (FIG. 3). The expression levels for Compound 1 alone at all doses were very low and thus not depicted in FIG. 3.

### Example 4: IFN-γ Secretion Assay

Expression/secretion of IFN-γ was induced in CMV-specific T-cells when stimulated with CMV antigen. B lymphoblastoid cells (B-LCL) were used as antigen presenting cells for the assay. A portion of these were used as a negative control and another portion were incubated with a peptide sequence from the CMV pp65 protein to serve as the positive control. After incubation to allow peptide uptake and binding to HLA-A*0201, the cells were washed to remove excess peptide. These cells were added to wells of U bottom 96 well plate at 20,000 cells per well. Antigen (CMV) specific T cells were added to all wells at 20,000 cells per well and test compounds were added at desired concentrations. The cultures were incubated for 24 hours after which a 100 μL portion culture medium were removed from each well. IFNγ concentration of these samples were measured using an immunoassay from Meso Scale Discovery. All conditions were prepared in triplicate.

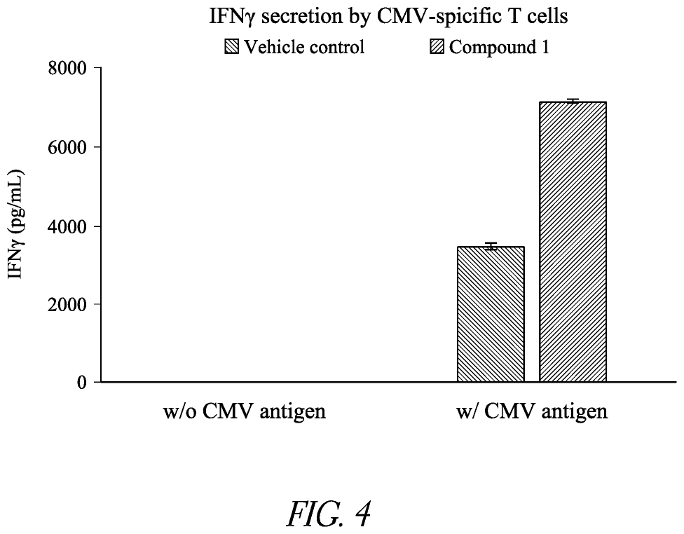

Treatment with Compound 1 enhances the levels of IFN-γ observed. No IFN-γ is observed without CMV peptide treatment, or when cells are treated with only Compound 1. Supernatants were harvested and ELISA was performed to measure soluble IFN-γ. The results are shown in FIG. 4.

### Example 5: Restoration of Cytotoxic T Cell Activity

This assay was used to assess the ability of a test compound to restore T cell function in vivo. Two antigen-specific cancer models were devised to assess the ability of test compounds to reverse exhaustion of tumor-reactive T cells. B16F10 melanoma was generated that express the full-length glycoprotein (GP) of LCMV. This facilitates the tracking of antigen-specific T cells. These cell lines have been validated to have similar invasiveness and growth patterns as their wild type counterparts.

500,000 B16F10 murine melanoma cells were injected subcutaneously into the hind flanks of 8-week old C57/BL6 mice. After tumors reached an average size of 35 mm², 25 μg of 1 mg/mL Compound 1 or vehicle (DMSO, 25 μL) was injected intra-tumorally. Mouse health and tumor size was monitored daily.

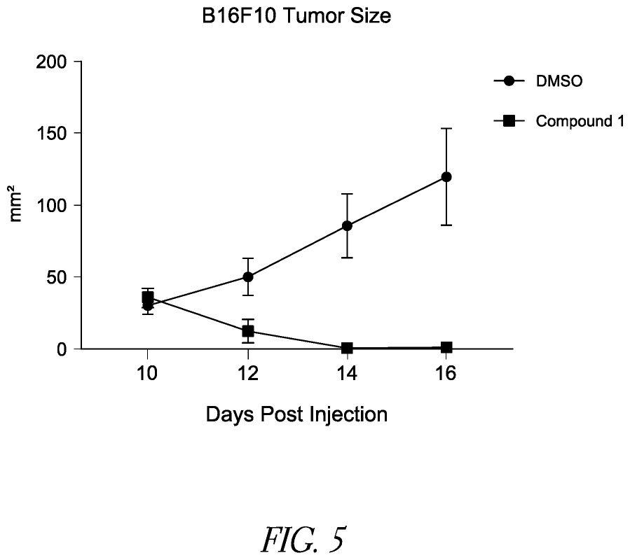

Intratumoral injection of Compound 1 resulted in decreased tumor size while injection of DMSO did not impede tumor growth (FIG. 5). Complete tumor regression was observed in 4 out of 5 mice. Marked immune response was observed at the site of injection.

### Example 6: Adoptive Transfer

To assess whether test compounds can rescue exhausted cells ex vivo, exhausted T cells (Tₑₓ) will be harvested from clone-13 infected mice 15 days post-infection. Tₑₓ cells will then be treated with test compound or vehicle for various durations (2, 4, 6 and 8 hrs), thoroughly washed and adoptively transferred in clone-13 infected congenic hosts (Infected for 15 days). Virus-specific T cell numbers and function will then be measured 5-7 days post infection as described above. Compounds deemed to rescue T cell exhaustion in the LCMV model in the syngeneic tumor models where LCMV glycoprotein is expressed.

### Example 7: Reversal of T Cell Exhaustion Ex Vivo

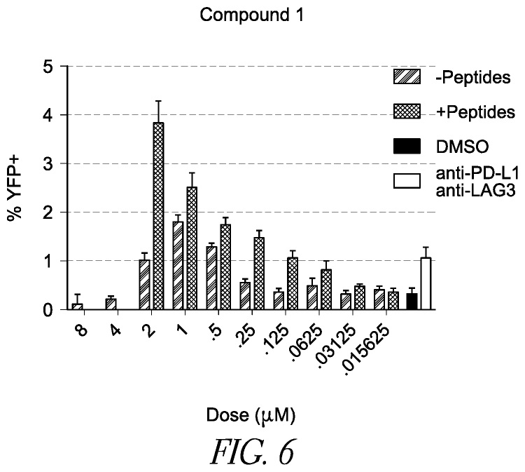

IFN-γ-YFP mice [Strain: C.129S4(B6)] were obtained from Jackson laboratory and infected with 2×10⁶ PFU lymphocytic choriomeningitis virus (LCMV) Clone 13. At day 15 post-infection, splenocytes were harvested and cultured with 1 μg/ml of a peptide mix of H-2^{b} immunodominant CD8 CTL epitopes (GP₃₃₋₄₁, GP₂₇₆₋₂₈₆, and NP₃₉₆₋₄₀₄) and the immunodominant CD4 T cell epitope (GP₆₇₋₈₀) for 3 days to provide T cell stimulation. In parallel, anti-PD-L1 or anti-PD-L1 and anti-LAG3 combinatorial antibody treatment were used as a positive control for restoration of T cell function. Compound 1 was then added to wells in the absence or presence of anti-PD-L1 to probe for synergistic effects with checkpoint blockade. After 5 days, YFP expression was assessed in T cells by flow-cytometry. Supernatants from wells incubated with Compound 1 were also evaluated for the compounds ability to restore TNF-α and IL-2 production from virus specific CD4 and CD8 T cells as described in Teijaro et al, Science 2013, 340(6129):207-211 and Walsh et al. Cell Host & Microbe 2012, 11, 643-653. The results are summarized in FIG. 6.

### Example 8: Reversal of T Cell Exhaustion Ex Vivo

Serum titers from LCMV-CL13 infected IFN-γ-YFP mice were measured by plaque assay at day 12 post infection (p.i.) to confirm a productive infection. At day 15 p.i., spleens were harvested, digested and single cell suspensions prepared using a mixture of collagenase/Dnase (Roche) prior to homogenation on a 100 μM filter using a butt-end of a syringe. Red blood cells (RBC) were lysed for 2 minutes per spleen in IX RBC lysis buffer. Following RBC lysis, B cells were depleted by magnetic bead separation using a CD19-positive selection II kit (EasySep). Splenocytes were counted and resuspended to 1×106 cells/mL in complete T cell media (10% FBS, 1% PenStrep, 1% L-Glutamine, NEAA, Sodium Pyruvate, HEPES, 50 μM BME) supplemented with 2 μg/mL LCMV-specific CD8 peptides (GP₃₃₋₄₁, NP₃₉₆₋₄₀₄ and GP₂₇₆₋₂₈₆) and 5 μg/mL CD4 peptide (GP₆₁₋₈₀). Next, 50 μL cells were seeded into 384-well flat-clear bottom TC-treated plates (Greiner, Cat #781090) that were pre-spotted using an Echo Liquid Handler (Labcyte) with dimethyl sulfoxide (DMSO) or a compound disclosed herein at 10 μM final concentration. Plates were placed at 37° C.+5% CO₂ at a 20° angle to increase cell to cell contact. Following a 5-day incubation period, 7-aminoactinomycin D was added to each well (1:50 dilution) and plates were rested for 15 minutes. Cells were then analyzed on a ZE5 flow cytometer (Bio-Rad).

A median absolute deviation (MAD) plate-based z-score for the frequency of YFP⁺7AAD⁻ cells in experimental wells compared to DMSO control wells was established for each 384-well plate and distribution of experimental z-scores was compared to a normal distribution (Table 1). Statistical analysis was performed using cellHTS2 version 2.46.0 and R version 3.5.2.

TABLE 1
Reversal of T-cell Exhaustion for Selected Compounds as
Measured by Z-Score

| | Mean |
|---|---|
| | Z-score at |
| Compound | 1 μM |
| 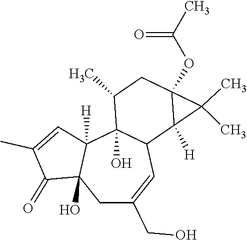 prostratin | 48.9 |
| 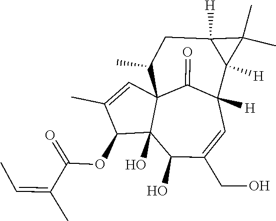 [SMILES: C/C=C(/C)C(=O)O[C@H]1C(C)=C[C@]23C(=O)[C@@H](C=C(CO)[C@@H](O)[C@]12O)[C@H]1[C@@H](C[C@H]3C)C1(C)C] Ingenol-3-angelate | 30.9 |
| 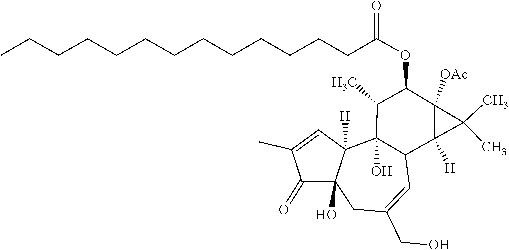 12-O-tetradecanoylphorbol-13-acetate | 17.8 |
| 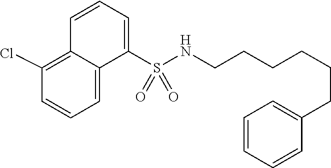 [SMILES: O=S(=O)(NCCCCCCc1ccccc1)c1cccc2c(Cl)cccc12] 5-chloro-N-(6-phenylhexyl)naphthalene-1-sulfonamide | 11.1 |
| 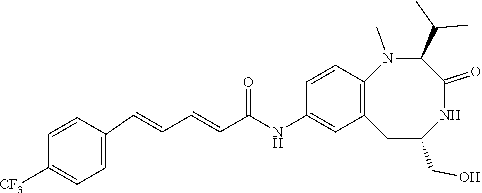 [SMILES: CC(C)[C@H]1C(=O)N[C@H](CO)Cc2cc(NC(=O)/C=C/C=C/c3ccc(C(F)(F)F)cc3)ccc2N1C] TPPB | 4.6 |
| 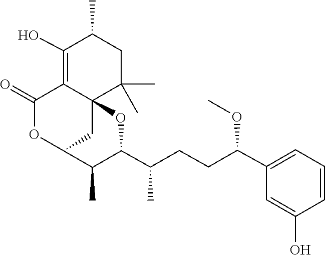 [SMILES: CO[C@@H](CC[C@H](C)[C@H]1O[C@@]23C[C@H](OC(=O)C2=C(O)[C@H](C)CC3(C)C)[C@@H]1C)c1cccc(O)c1] Neo-debromoaplysiatoxin B | 3.6 |
| 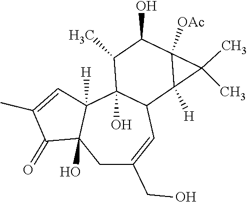 Phorbol-13-acetate | 0.9 |
| 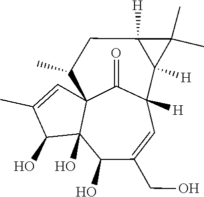 Ingenol | 0.6 |

### Example 9: Induction of CD69 Expression

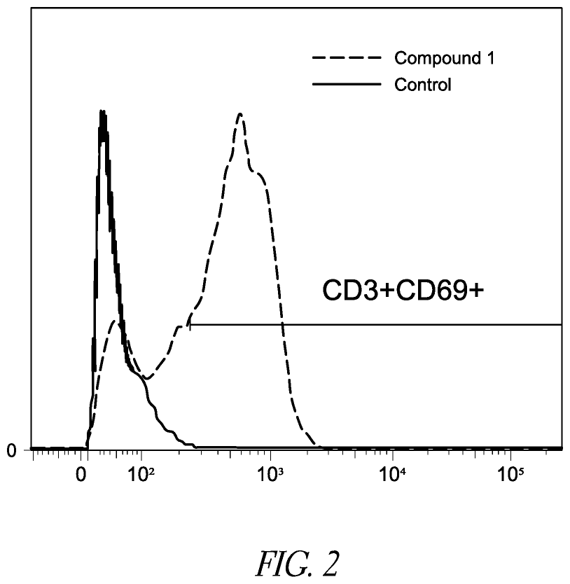

Expression of CD69 is induced in PBMCs by treatment with Compound 1. Co-stimulation of TCR positive cells was accomplished with immobilized anti-CD3 antibody. The experimental procedure may be performed as described in Trickett, A. et. al. J. Immunol. Methods, 2003, 275, 251-255, the entirety of which is incorporated by reference herein. Cell population was gated on CD3 (TCR positive population), allowing CD69 expression on T-cells to be observed (FIG. 2).

### Example 10: Induction of T Cell Activity and Infiltration in the Tumor Microenvironment

This assay was used to assess the ability of a test compound to induce T cell activity and infiltration in vivo.

500,000 B16F10 murine melanoma cells were injected subcutaneously into the hind flanks of 8-week old C57/BL6 mice. After tumors reached an average size of 35 mm², 0.25 μg and 0.05 ug of 1 mg/mL Compound 1 or vehicle (DMSO, 25 μL) was injected intra-tumorally. The cellular population of the tumor infiltrate was monitored at a single time point using flow cytometry methods.

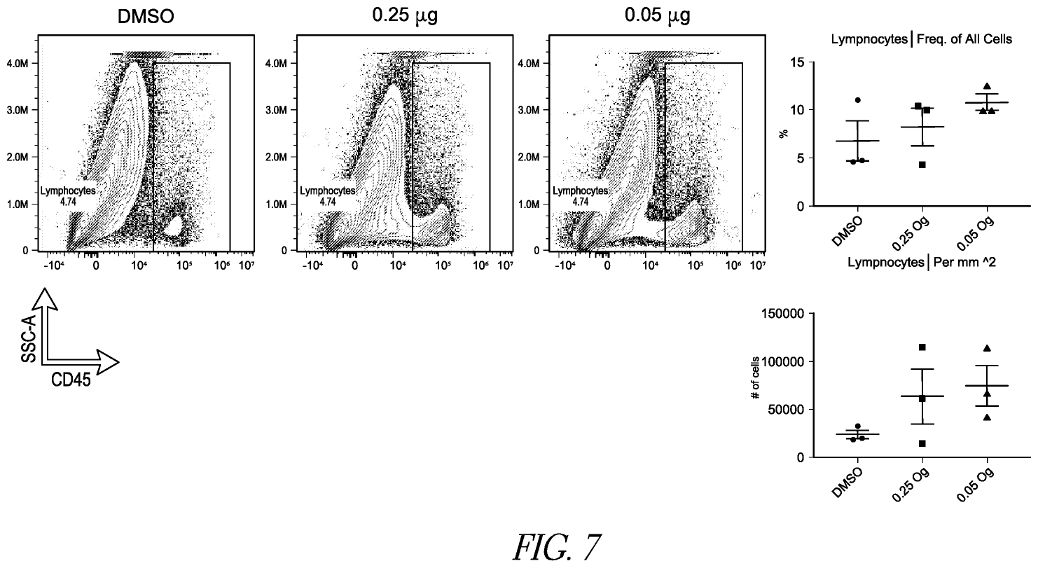

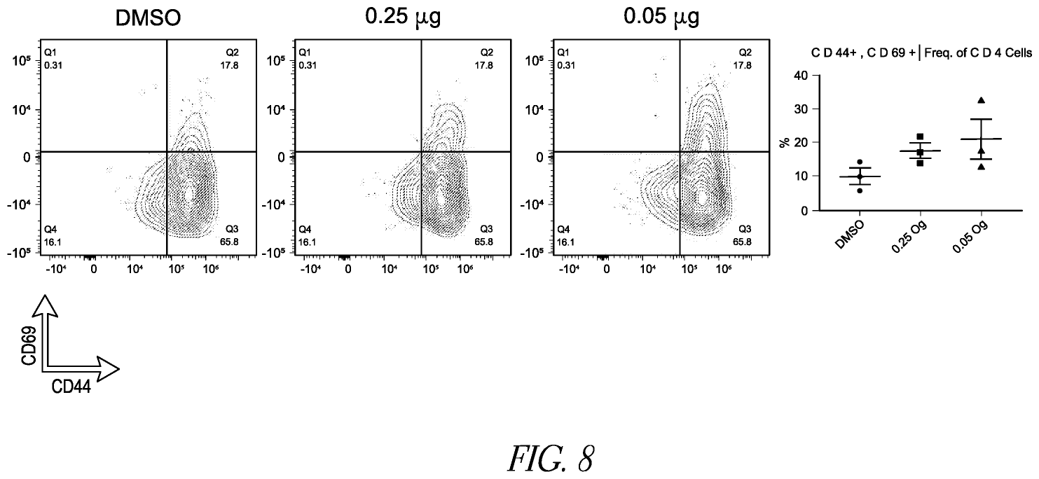

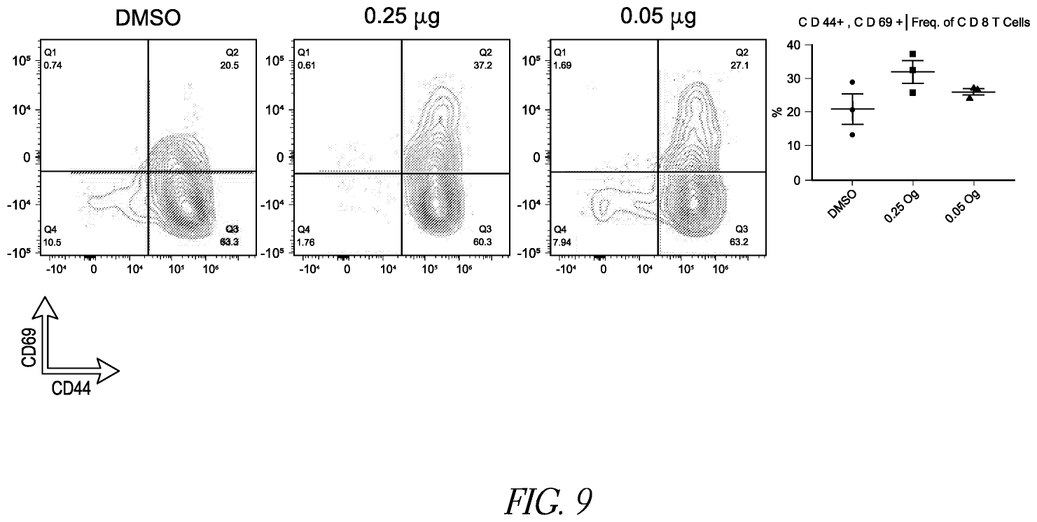

Intra-tumoral injection of Compound 1 resulted in increased lymphocyte infiltrate versus DMSO control (FIG. 7). Intratumoral injection of Compound 1 resulted in increased CD4+ CD69+ T cell infiltrate versus DMSO control (FIG. 8). Intratumoral injection of Compound 1 resulted in increased CD8+ CD69+ T cell infiltrate versus DMSO control (FIG. 9).

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A method of reversing T-cell exhaustion in a subject, the method comprising administering to the subject a compound of Formula (I): 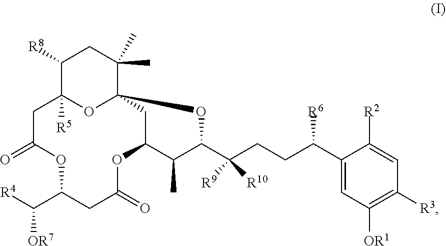
or a pharmaceutically acceptable salt or prodrug thereof, wherein:
R¹ is —H, —C(═O)CH₃, —CH₂C₆H₅, —CH₂—O—CH₂C₆H₅, C(═O)NHCH₃, or —CH₃;
R² is —H, —OH, —CH₃, -halo, —OC(═O)CH₃, —NHC(═O)CH₃, or —NO₂;
R³ is —H, —OH, —CH₃, -halo, —OC(═O)CH₃, —NHC(═O)CH₃, or —NO₂;
R⁴ is —H or —CH₃;
R⁵ is —H, —OMe or —OH;
R⁶ is —H, —OCH₃, or -halo;
R⁷ is —H, —C(═O)CH₃, —CH₂C₆H₅, —CH₂—O—CH₂C₆H₅, —C(═O)O—C₆H₅OH, —C(═O)NHCH₃, or —CH₃;
R⁸ is —H or —CH₃;
R⁹ is —H or —CH₃; and
R¹⁰ is —H or —CH₃.

2. The method of claim 1, wherein R¹, R², and R³ are each —H.

3. The method of claim 1 or claim 2, wherein R⁴ is CH₃.

4. The method of any one of claims 1 to 3, wherein R⁵ is —OH.

5. The method of any one of claims 1 to 3, wherein R⁵ is —H.

6. The method of any one of claims 1 to 5, wherein R⁶ is —H.

7. The method of any one of claims 1 to 5, wherein R⁶ is —OCH₃.

8. The method of any one of claims 1 to 7, wherein R⁷ is —H.

9. The method of any one of claims 1 to 8, wherein R⁸ is —H.

10. The method of any one of claims 1 to 8, wherein R⁸ is —CH₃.

11. The method of any one of claims 1 to 10, wherein R⁹ is —H.

12. The method of any one of claims 1 to 10, wherein R⁹ is —CH₃.

13. The method of any one of claims 1 to 12, wherein R¹⁰ is —H.

14. The method of any one of claims 1 to 12, wherein R₁₀ is —CH₃.

15. The method of claim 1, wherein the compound is selected from the group consisting of: 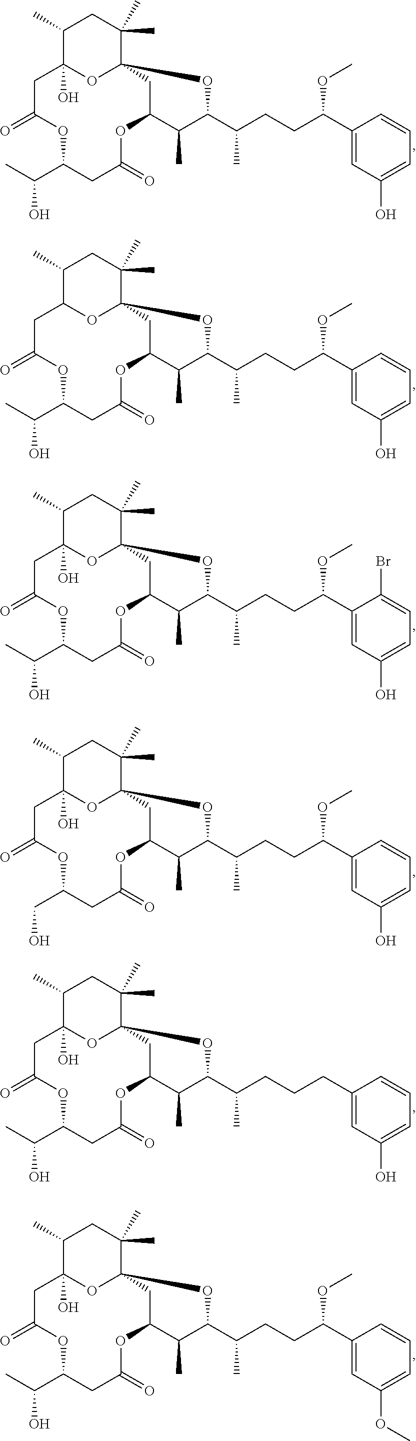
and pharmaceutically acceptable salts and prodrugs thereof.

16. A method of reversing T-cell exhaustion in a subject, the method comprising administering to the subject a compound having the structure: 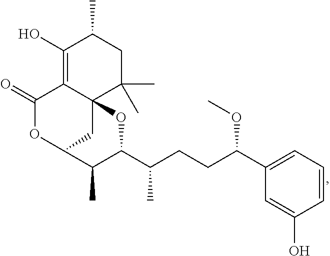 [SMILES: CO[C@@H](CC[C@H](C)[C@H]1O[C@@]23C[C@H](OC(=O)C2=C(O)[C@H](C)CC3(C)C)[C@@H]1C)c1cccc(O)c1]
or a pharmaceutically acceptable salt or prodrug thereof.

17. A method of reversing T-cell exhaustion in a subject, the method comprising administering to the subject a Protein kinase C (PKC) theta activator.

18. The method of claim 17, wherein the PKC theta activator is a phorbol ester

19. The method of claim 11, wherein the phorbol ester is 12-O-tetradecanoylphorbol-13-acetate, 12-deoxyphorbol-13-phenylacetate, prostratin, or phorbol-13 acetate.

20. The method of claim 17, wherein the PKC theta activator is a teleocidin.

21. The method of claim 20, wherein the teleocidin is selected from the group consisting of: teleocidin A-1, teleocidin A-2, teleocidin B-1, teleocidin B-2, teleocidin B-3, teleocidin B-4, teleocidin B-18, des-O-methylolivoretin C, des-N-methylteleocidin B-4, blastmycetin A, blastnycetin B, blastmycetin C, blastmycetin D, blastmycetin E, blastmycetin F, (−)-indolactan-V, (−)-14-O-malonylindolactam-V, (−)-14-O-acetylindolactam-V, (−)-7-geranylindolactam-V, N13-desmethylteleocidin A-1, N13-desmethylteleocidin B-4, (−)-2-oxy-indolactam, olivoretin A (14-O-methylteleocidin B), olivoretin B, and olivoretin C, olivoretin A, olivoretin B, olivoretin C, olivoretin D, olivoretin E, des-O-methylolivoretin C, pendolmycin, 14-O—(N-acetylglucosaminyl)teleocidin, and (2E,4E)-N-((2S,5S)-5-(hydroxymethyl)-2-isopropyl-1-methyl-3-oxo-1,2,3,4,5,6-hexahydrobenzo[e][1,4]diazocin-8-yl)-5-(4-(trifluoromethyl)phenyl)penta-2,4-dienamide.

22. The method of claim 17, wherein the PKC theta activator is ingenol, ingenol-3-angelate, or ingenol-3-dodecanoate.

23. The method of claim 17, wherein the PKC theta activator is farnesyl thiotriazole, 2-[(2-pentylcyclopropyl)methyl]cyclopropaneoctanoic acid, or 5-chloro-N-(6-phenylhexyl)naphthalene-1-sulfonamide).

24. The method of claim 17, wherein the PKC theta activator is a bryostatin.

25. The method of claim 24, wherein the bryostatin is bryostatin-1, bryostatin-2, bryostatin-3, bryostatin-4, bryostatin-5, bryostatin-6, bryostatin-7, bryostatin-8, or bryostatin-9.

26. The method of claim 17, wherein the PKC theta activator is

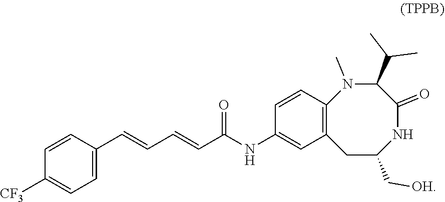 [SMILES: CC(C)[C@H]1C(=O)N[C@H](CO)Cc2cc(NC(=O)/C=C/C=C/c3ccc(C(F)(F)F)cc3)ccc2N1C]

27. The method of any one of claims 1 to 26, comprising administering to the subject a pharmaceutical composition comprising the compound and one or pharmaceutically acceptable excipients.

28. The method of any one of claims 1 to 27, further comprising administering to the subject a second active agent.

29. The method of claim 28, wherein the compound of Formula (I) and the second active agent are administered simultaneously.

30. The method of claim 28, wherein the compound of Formula (I) and the second active agent are administered sequentially.

31. The method of any one of claims 1 to 30, wherein the subject has cancer.

32. The method of claim 31, wherein the cancer is acute lymphoblastic leukemia, acute myeloid leukemia, bladder cancer, breast cancer, Burkitt lymphoma, cervical cancer, chronic lymphocytic leukemia, chronic myelogenous leukemia, colorectal cancer, esophageal cancer, Ewing sarcoma, gastric cancer, testicular cancer, renal cancer, hepatocellular cancer, melanoma, multiple myeloma, neuroblastoma, Hodgkin's lymphoma, non-Hodgkin lymphoma, pancreatic cancer, prostate cancer, rectal cancer, or thyroid cancer.

33. The method of any one of claims 1 to 32, wherein the administration is oral, intravenous, intraperitoneal, intragastric, or intravascular.

34. The method of any one of claims 1 to 32, wherein the administration is by intratumoral injection.

35. A method of reversing T-cell exhaustion in a subject, the method comprising the steps of:
(i) obtaining a biological sample comprising T-cells from the subject;
(ii) contacting said T-cells with a compound of any one of claims 1 to 26; and
(iii) administering the T-cells contacted with said compound to said subject.

36. The method of claim 35, further comprising administering a second active agent to the subject.

37. A method of shrinking a tumor in a subject, the method comprising administering to the subject a compound of Formula (I): 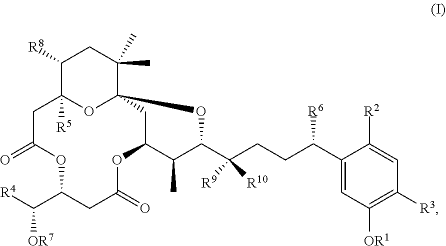
or a pharmaceutically acceptable salt or prodrug thereof, wherein:
R¹ is —H, —C(═O)CH₃, —CH₂C₆H₅, —CH₂—O—CH₂C₆H₅, C(═O)NHCH₃, or —CH₃;
R² is —H, —OH, —CH₃, -halo, —OC(═O)CH₃, —NHC(═O)CH₃, or —NO₂;
R³ is —H, —OH, —CH₃, -halo, —OC(═O)CH₃, —NHC(═O)CH₃, or —NO₂;
R⁴ is —H or —CH₃;
R⁵ is —H, OMe or —OH;
R⁶ is —H, —OCH₃, or -halo;
R⁷ is —H, —C(═O)CH₃, —CH₂C₆H₅, —CH₂—O—CH₂C₆H₅, —C(═O)O—C₆H₅OH, —C(═O)NHCH₃, or —CH₃;
R⁸ is —H or —CH₃;
R⁹ is —H or —CH₃; and
R¹⁰ is —H or —CH₃.

38. A method of shrinking a tumor in a subject, the method comprising administering to the subject a compound having the structure: 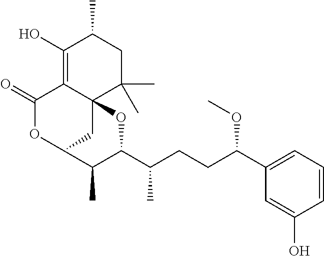 [SMILES: CO[C@@H](CC[C@H](C)[C@H]1O[C@@]23C[C@H](OC(=O)C2=C(O)[C@H](C)CC3(C)C)[C@@H]1C)c1cccc(O)c1]
or a pharmaceutically acceptable salt or prodrug thereof.

39. A method of shrinking a tumor in a subject, the method comprising administering to the subject comprising administering to the subject a Protein kinase C (PKC) theta activator.

40. The method of claim 39, wherein the PKC theta activator is a phorbol ester

41. The method of claim 40, wherein the phorbol ester is 12-O-tetradecanoylphorbol-13-acetate or 12-deoxyphorbol-13-phenylacetate, prostratin, or phorbol-13 acetate.

42. The method of claim 39, wherein the PKC theta activator is a teleocidin.

43. The method of claim 42, wherein the teleocidin is selected from the grup consisting of: teleocidin A-1, teleocidin A-2, teleocidin B-1, teleocidin B-2, teleocidin B-3, teleocidin B-4, teleocidin B-18, des-O-methylolivoretin C, des-N-methylteleocidin B-4, blastmycetin A, blastmycetin B, blastmycetin C, blastmycetin D, blastmycetin F, blastmycetin F, (−)-indolactarn-V, (−)-14-O-malonylindolactam-V, (−)-14-O-acetylindolactam-V, (−)-7-geranylindolactam-V, N13-desmethylteleocidin A-1, N13-desmethylteleocidin B-4, (−)-2-oxy-indolactam, olivoretin A (14-O-methylteleocidin B), olivoretin B, and olivoretin C, olivoretin A, olivoretin B, olivoretin C, olivoretin D, olivoretin E, des-O-methylolivoretin C, pendolmycin, 14-O—(N-acetylglucosaminyl)teleocidin and (2E,4E)-N-((2S,5S)-5-(hydroxymethyl)-2-isopropyl-1-methyl-3-oxo-1,2,3,4,5,6-hexahydrobenzo[e][1,4]diazocin-8-yl)-5-(4-(trifluoromethyl)phenyl)penta-2,4-dienamide.

44. The method of claim 39, wherein the PKC theta activator is ingenol, ingenol-3-angelate, or ingenol-3-dodecanoate.

45. The method of claim 39, wherein the PKC theta activator is farnesyl thiotriazole, 2-[(2-pentylcyclopropyl)methyl]cyclopropaneoctanoic acid, or 5-chloro-N-(6-phenylhexyl)naphthalene-1-sulfonamide).

46. The method of claim 39, wherein the PKC theta activator is a bryostatin.

47. The method of claim 46, wherein the bryostatin is bryostatin-1, bryostatin-2, bryostatin-3, bryostatin-4, bryostatin-5, bryostatin-6, bryostatin-7, bryostatin-8, or bryostatin-9.

48. The method of claim 39, wherein the PKC theta activator is

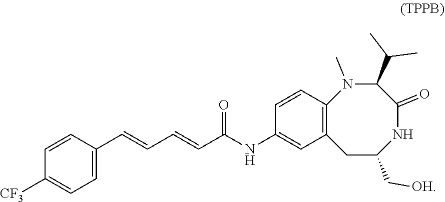 [SMILES: CC(C)[C@H]1C(=O)N[C@H](CO)Cc2cc(NC(=O)/C=C/C=C/c3ccc(C(F)(F)F)cc3)ccc2N1C]

49. The method of any one of claims 39-48, wherein the administration is oral, intravenous, intraperitoneal, intragastric, or intravascular.

50. The method of any one of claims 39-48, wherein the administration is by intratumoral injection.

51. The method of any one of claims 39-49, wherein the tumor shrinks by 10%.

52. The method of any one of claims 39-49, wherein the tumor shrinks by 20%.

53. The method of any one of claims 39-49, wherein the tumor shrinks by 30%.

54. The method of any one of claims 39-49, wherein the tumor shrinks by 50%.

55. The method of any one of claims 39-49, wherein the tumor shrinks by 70%.

56. A method of treating cancer, comprising administering a compound of any one as described in claims 1 to 26 to a subject in need thereof by intratumoral administration.

57. A method of improving the anti-tumor activity of a T cell, the method comprising administering a therapeutically effective amount of a pharmaceutical composition comprising a compound as described in any one of claims 1-26 to the subject.

58. A method of improving, the anti-tumor activity of a T cell, the method comprising:
obtaining a biological sample comprising T-cells from the subject;
contacting said T-cells with a compound as described in any one of claims 1 to 26; and
administering the T-cells contacted with said compound to said subject.

59. A method of inducing a NFAT-dependent T cell activation in a subject, said method comprising administering a therapeutically effective amount of a pharmaceutical composition comprising a compound as described in any one of claims 1-26 to the subject.

60. The method of any one of claims 56 to 59, wherein the subject has infectious disease.

61. The method of any one of claims 56 to 60, wherein the subject has a viral infection.

62. The method of claim 61, wherein the viral infection is HIV.

63. A method of increasing the pool of immune checkpoint inhibitor responsive T cells in the tumor microenvironment comprising administering a therapeutically effective amount of a pharmaceutical composition comprising a compound as described in any one of claims 1-26 to the subject.

64. A method of increasing lymphocyte infiltration in the tumor microenvironment comprising administering a therapeutically effective amount of a pharmaceutical composition comprising a compound as described in any one of claims 1-26 to the subject.

65. A method of increasing the population of activated CD4+ and/or CD8+ cells in the tumor microenvironment comprising administering a therapeutically effective amount of a pharmaceutical composition comprising a compound as described in any one of claims 1-26 to the subject.
